(19) United States
(12) Patent Application Publication
(10) Pub. No.: US 2003/0147899 A1
(43) Pub. Date: Aug. 7, 2003

(54) Endogenous hormone adjuvant and uses thereof

(76) Inventor: Firdaus S. Dhabhar, Columbus, OH (US)

Correspondence Address: KLAUBER & JACKSON, 411 HACKENSACK AVENUE, HACKENSACK, NJ, 07601

(21) Appl. No.: 10/378,716

(22) Filed: Mar. 4, 2003

Related U.S. Application Data
(63) Continuation of application No. 09/488,132, filed on Jan. 20, 2000.
(60) Provisional application No. 60/116,919, filed on Jan. 22, 1999.

(51) Int. Cl. A61K 39/38; A61K 39/12; A61K 39/02; A61K 31/56

(52) U.S. Cl. 424/184.1; 514/171; 424/204.1; 424/234.1

(57) ABSTRACT

This invention provides a hormone adjuvant composition comprising an amount of endogenous epinephrine, endogenous glucocorticoids, analogs thereof, and combinations thereof Further, this invention provides a vaccine comprising an amount of endogenous epinephrine and endogenous glucocorticoids and a suitable carrier or diluent. This invention provides a method of stimulating or enhancing an antigen-specific cell-mediated immune response; a method for conferring protection against an infectious agent; and a method of immunomodulation as treatment in a subject with an infectious agent or cancer comprising administering a low dose of the hormone adjuvant composition.

### FIELD OF THE INVENTION

This invention provides an endogenous hormone adjuvant composition comprising an amount of an endogenous epinephrine, endogenous glucocorticoids, and combinations thereof. This invention further provides a hormone adjuvant composition comprising an analog of epinephrine or glucocorticoids, natural or synthetic, or combinations thereof Further, this invention provides a vaccine comprising an amount of endogenous epinephrine, endogenous glucocorticoids, or analogs thereof, and combinations thereof Lastly, this invention provides a method of stimulating or enhancing an antigen-specific cell-mediated immune response; a method for conferring protection against an infectious agent; and a method of treating a subject with an infectious agent or cancer comprising administering a low dose of the hormone adjuvant composition.

### BACKGROUND OF THE INVENTION

Stress is a familiar aspect of modern life, being a stimulant for some, but a concern for many. Stress has long been suspected to play a role in the etiology of many diseases, and numerous studies have shown that stress can be immuno-suppressive and hence may be detrimental to health (2-10). Moreover, glucocorticoid stress hormones are widely regarded as being immuno-suppressive (2), and are used clinically as anti-inflammatory agents (11). However, a suppression of immune function under all stress conditions would not be evolutionarily adaptive. Stress is an intrinsic part of life for most organisms, and dealing successfully with stressors is what enables survival. Environmental challenges and most evolutionary selection pressures, are stressors which may be psychological (fear, anxiety), physical (wounding, infection), or physiological (food or water deprivation). One of the primary functions of the brain is to perceive stress and to warn and enable an organism to deal with its consequences. For example, when a gazelle sees a charging lion, the gazelle's brain detects a threat and orchestrates a physiologic response which first prepares, and then enables, the gazelle to flee. Under such circumscribed stress conditions, just as the stress response prepares the nervous, cardiovascular, musculoskeletal, and neuroendocrine systems for fight or flight, it may also prepare the immune system for challenges (e.g. wounding or infection) which may be imposed by the stressor (1, 12-15).

Initial studies in rats showed that acute stress (2 h restraint) results in a significant redistribution of leukocytes from the blood to other organs (skin, lymph nodes, bone marrow) in the body (12, 13, 17), and that adrenal stress hormones are the major mediators of this leukocyte redistribution (14). The skin and certain lymph nodes were identified as targets to which immune cells trafficked during stress (15, 17). The two major stress hormones, epinephrine and corticosterone, have been identified as mediators of the stress-induced redistribution of immune cells (14).

Vaccine adjuvants are useful for improving an immune response obtained with any particular antigen in a vaccine composition. Adjuvants are used to increase the amount of antibody and effector T cells produced and to reduce the quantity of antigen and the frequency of injection. Although some antigens are administered in vaccines without an adjuvant, there are many antigens that lack sufficient immunogenicity to stimulate a useful immune response in the absence of an effective adjuvant. Adjuvants also improve the immune response from “self-sufficient” antigens, in that the immune response obtained may be increased or the amount of antigen administered may be reduced.

The standard adjuvant for use in laboratory animals is Freund's adjuvant. Freund's Complete adjuvant (FCA) is an emulsion containing mineral oil and killed mycobacteria in saline. Freund's incomplete adjuvant (FIA) omits the mycobacteria. Both FIA and FCA induce good humoral (antibody) immunity, and FCA additionally induces high levels of cell-mediated immunity. However, neither FCA nor FIA are acceptable for clinical use due to the side effects. In particular, mineral oil is known to cause granulomas and abscesses, and Mycobacterium tuberculosis is the agent responsible for tuberculosis.

There have been many substances that have been tried to be used as adjuvants, such as the lipid-A portion of gram negative bacterial endotoxin, and trehalose dimycolate of mycobacteria. The phospholipid lysolecithin exhibited adjuvant activity (Arnold et al., Eur. J Immunol. 9:363-366, 1979). Some synthetic surfactants exhibited adjuvant activity, including dimethyldioctadecyl ammonium bromide (DDA) and certain linear polyoxypropylenepolyoxyethylene (POP-POE) block polymers (Snippe et al., Int. Arch.

Allergy Appl. Immunol. 65:390-398, 1981; and Hunter et al., J. Immunol. 127:1244-1250, 1981) While these natural or synthetic surfactants demonstrate some degree of adjuvant activity, they do not demonstrate the degree of immunopotentiation (i.e., adjuvant activity) as FCA or FIA.

### SUMMARY OF THE INVENTION

This invention provides an endogenous hormone adjuvant composition comprising an amount of endogenous epinephrine, endogenous glucocorticoids, or analogs thereof, and combinations thereof This invention provides a pharmaceutical comprising the endogenous hormone adjuvant composition comprising an amount of endogenous epinephrine (Adrenaline) and endogenous glucocorticoids, or analogs thereof, and a suitable carrier or diluent.

This invention provides a vaccine comprising an amount of endogenous epinephrine and endogenous glucocorticoids, or analogs thereof, and a suitable carrier or diluent.

This invention provides a therapeutic composition, comprising a mixture of a therapeutically effective antigen or vaccine and a hormone adjuvant composition of endogenous epinephrine and endogenous glucocorticoids, or analogs thereof, and a suitable carrier or diluent.

This invention provides a method of stimulating or enhancing an antigen-specific cell-mediated immune response which comprises administering to a subject an amount of an immunomodulator as a vaccine and a low dose of the hormone adjuvant composition comprising an amount of endogenous epinephrine, endogenous glucocorticoids, or analogs thereof, and combinations thereof.

This invention provides a method for conferring protection against an infectious agent which comprises administering to a, subject an amount of an immunomodulator as a vaccine and a low dose of the composition in an amount of endogenous epinephrine and endogenous glucocorticoids, or analogs thereof, and a suitable carrier or diluent.

This invention provides a method of treating a subject with an infectious disease or agent, or cancer comprising administering to a subject an amount of a low dose of the hormone adjuvant composition as an immunomodulator comprising an amount of endogenous epinephrine, endogenous glucocorticoids, or analogs thereof, or combinations thereof, and a suitable carrier or diluent.

### BRIEF DESCRIPTION OF THE FIGURES

FIG. 1A-C EHA-Induced Enhancement of Cell-Mediated Immunity.

The DTH response of four groups of animals is compared. One group of animals, the control group, was injected with vehicle (VEH) at the time of immunization with antigen (1.5% Oxazolone, OXA) (A, B, C). A second group was injected with epinephrine (0.5 mg/kg) (A) at the time of immunization, a third group with corticosterone (5 mg/kg) (B) at the time of immunization, and a fourth group with a “hormone cocktail” consisting of a low-dose formulation of epinephrine and corticosterone (epinephrine (0.5 mg/kg)+corticosterone (5 mg/kg))(C). The strength of a cell-mediated immune response or DTH reaction mounted against OXA was examined six days after immunization and hormone administration by challenging the skin (dorsal aspect of ear) with a low concentration of OXA.

FIG. 2 Effects of stress on the DTH response of INTACT (A), SHAM (B), and ADX (C) animals. A six day timecourse of changes in thickness of right pinnae of previously sensitized animals challenged with DNFB (0.5% w/v) is shown. Stressed INTACT and SHAM animals showed a significant increase in the DTH response compared to unstressed animals. ADX animals did not show a stress-induced increase skin DTH. Data are expressed as means±SEM (n=6 per treatment group). Statistically significant differences are indicated: * p<0.05, ** p<0.005, independent t test.

FIG. 3 Acute administration of corticosterone enhances skin DTH. Corticosterone (CORT, 5 mg/kg) was administered (ip) to ADX animals (A) or through drinking water (100 or 400 μ/ml) to adrenal intact animals (B). Control animals were treated with vehicle (30% HBC (2A) or 0.6% ethanol (2B)). Corticosterone treated animals showed a significantly larger DTH response than vehicle treated animals. (* p<0.05, independent t test).

FIG. 4 Pharmacologic treatment with glucocorticoid hormones suppresses the skin DTH response. A timecourse of changes in thickness of right pinnae of previously sensitized animals challenged with DNFB (0.5% w/v) is shown. Corticosterone (CORT, 40 mg/kg) or dexamethasone (DEX, 0.1 mg/kg) were administered acutely (A). Corticosterone was also administered chronically in drinking water (400 μg/ml, 6 days) (B). Control animals were treated with vehicle (30% HBC, FIG. 4A; or 0.6% ethanol, FIG. 4B). Corticosterone and dexamethasone treated animals showed lower DTH responses than control animals. (* p<0.05, ** p<0.005, independent t test).

FIG. 5 Acute administration of epinephrine enhances skin DTH. A timecourse of changes in thickness of right pinnae of previously sensitized animals challenged with DNFB (0.5% w/v) is shown. Epinephrine (0.05. 0.25, or 0.5 mg/kg) was administered acutely to ADX animals. Control animals were treated with vehicle (ddH₂O). Epinephrine treated animals showed a dose dependent increase in skin DTH. (* p<0.05, ** p<0.005, independent t test).

FIG. 6 Epinephrine and corticosterone additively enhance skin DTH. A timecourse of changes in thickness of right pinnae of previously sensitized animals challenged with OXA (0.75% w/v) is shown. Epinephrine (0.5 mg/kg), corticosterone (5 mg/kg) or epinephrine+corticosterone (EPI 0.5 mg/kg+CORT 5 mg/kg) were administered acutely to ADX animals. Control animals were treated with vehicle (30% HBC). Epinephrine or corticosterone treated animals showed an enhanced DTH response. Moreover, simultaneous administration of the two hormones resulted in an additive enhancement of skin DTH. (* p<0.05, ** p<0.005, independent t test).

FIG. 7 Acute administration of stress hormones increases the cellularity of cervical lymph nodes with drain the site of the skin DTH reaction. Epinephrine (0.5 mg/kg), corticosterone (5 mg/kg) or epinephrine+corticosterone (EPI 0.5 mg/kg+CORT 5 mg/kg) were administered to ADX animals. Control animals were treated with vehicle (30% HBC). Lymph nodes were collected and lymphocytes isolated 48 h after the induction of DTH. Compared to vehicle treated animals, hormone treated animals showed higher lymphocyte numbers in cervical lymph nodes which drain the site of the DTH reaction. (* p<0.05 independent t test).

### DETAILED DESCRIPTION OF THE INVENTION

The present invention provides a low-dose stress hormone “cocktail” (Endogenous Hormone Adjuvant, EHA) as a natural immuno-enhancing agent. An important, but unappreciated mechanism by which the body maintains health is the stress-response system. During any stressful event, the key messengers of this physiologic “red alert” are the stress hormones.

Stress is a familiar aspect of modern life, being a stimulant for some, but a concern for many. As defined herein “stress” is a constellation of events, which begins with a stimulus (stressor), which precipitates a reaction in the brain (stress perception), which subsequently activates physiologic systems in the body (stress response) (Dhabhar, F. S. and McEwen, B. S., Brain Behavior & Immunity 11:286-306 (1997). The physiologic stress response results in the release of neurotransmitters and hormones which serve as the brain's messengers to the rest of the body. An important distinguishing characteristic of stress is its duration and intensity. Thus, we define acute stress as stress that lasts for a period of a few minutes to a few hours, and chronic stress as stress that persists for several hours per day for several days. The intensity of stress may be gauged by the peak levels of stress hormones, neurotransmitters, and other physiological changes such as increases in heart rate and blood pressure, and by the amount of time for which these changes persist during and following stressor exposure. An important marker for deleterious levels of chronic stress may be a breakdown in the constancy of the circadian corticosterone rhythm (Dhabhar, F. S. and McEwen, B. S., Brain Behavior & Immunity 11:286-306 (1997); Sephton, et al., Psychoneuroimmunology Res. Soc. Abstract, No. S6.1, Boulder, Co. (1997). For,example, in one embodiment the stress level of Cortisol/corticosterone may be 20-45 microgm/100 ml and the level of Epinephrine 200-400 ng/l.

Acute stress is defined as stress that lasts (in terms of a significant elevation above baseline for: plasma glucocorticoid, plasma catecholamines, heart rate, and blood pressure) for a period of a few minutes to a few hours. Acute hormone treatment is defined as a quantity of hormone injected which produces stress levels of hormone in the plasma for a period of a few minutes to a few hours.

There are two important phases in the development of a cell-mediated immune response which is also known as a delayed type hypersensitivity (DTH) response. The first phase is the immunization or sensitization phase. During this phase, the organism is exposed to an antigen against which it establishes an immunologic memory. This phase is very similar to vaccination which involves introduction of an antigen (e.g. attenuated hepatitis) against which an individual mounts an immune response and forms an immunologic memory. The second phase is the challenge phase. During this phase, the organism is re-exposed to the initial immunizing antigen and as a result, it mounts a secondary immune response against the antigen. If the secondary response is primarily a cell-mediated immune response, the ensuing reaction is known as a DTH reaction. The challenge phase is similar to the immune reactions which would be mounted if an individual who is vaccinated against hepatitis is subsequently exposed to the virus. Having developed an immunologic memory for viral antigens (on account of the initial immunization) the individual's immune system now mounts a full-blown attack against the invading virus and eliminates it before the individual comes down with hepatitis.

The term “adjuvant” or “immunomodulator” refers to an agent, compound or mixture which is able to modulate the immune system or a particular immune response. Modulation may include, for instance the induction of movement of immune cells from one compartment of the body to another (e.g. from blood to skin or lymph nodes). Modulation may further include, for example, the enhancement of immune response, including antibody production, to an antigen. An adjuvant can serve as a tissue depot that slowly releases the antigen and also as a lymphoid system activator that non-specifically enhances the immune response (Hood et al., Immunology, Second Ed., 1984, Benjamin/Cummings: Menlo Park, Calif., p. 384). Often, a primary challenge with an antigen alone, in the absence of an adjuvant, will fail to elicit a humoral or cellular immune response. Examples of previously known and utilized adjuvants include, but are not limited to, complete Freund's adjuvant, incomplete Freund's adjuvant, saponin, mineral gels such as aluminum hydroxide, surface active substances such as lysolecithin, pluronic polyols, polyanions, peptides, oil or hydrocarbon emulsions, keyhole limpet hemocyanins, dinitrophenol, and potentially useful human adjuvant such as BCG (bacille Calmette-Guerin) and Corynebacterium parvum. Mineral salt adjuvants include but are not limited to: aluminum hydroxide, aluminum phosphate, calcium phosphate, zinc hydroxide and calcium hydroxide. Preferably, the adjuvant composition further comprises a lipid of fat emulsion comprising about 10% (by weight) vegetable oil and about 1-2% (by weight) phospholipids. Preferably, the adjuvant composition further optionally comprises an emulsion form having oily particles dispersed in a continuous aqueous phase, having an emulsion forming polyol in an amount of from about 0.2% (by weight) to about 49% (by weight), optionally a metabolizable oil in an emulsion-forming amount of up to 15% (by weight), and optionally a glycol ether-based surfactant in an emulsion-stabilizing amount of up to about 5% (by weight).

Preferably, the adjuvant or immunomodulator, as used and described as the invention herein, is an endogenous hormone adjuvant composition comprising endogenous epinephrine, endogenous glucocorticoids, or analogs thereof, and combinations thereof, which is pharmaceutically acceptable. In addition, the endogenous hormone adjuvant composition of the present invention may be combined with other previously known and utilized adjuvants.

The term “effective amount” of an immunomodulator refers to an amount of an immunomodulator sufficient to modulate an immune response, be it cell-mediated, humoral or antibody-mediated. An effective amount of an immunomodulator, if injected, can be in the range of about 0.1-1,000 .mu.g, preferably 1-900 .mu.g, more preferably 5-500 .mu.g, for a human subject, or in the range of about 0.01-10.0 .mu.g/Kg body weight of the subject animal. This amount may vary to some degree depending on the mode of administration, but will be in the same general range. If more than one immunomodulator is used, each one may be present in these amounts or the total amount may fall within this range. An effective amount of an antigen may be an amount capable of eliciting a demonstrable immune response in the absence of an immunomodulator. For many antigens, this is in the range of about 5-100 .mu.g for a human subject. Since the vaccines of the invention utilize an immunomodulator (in this case EHA) which enhances the natural immune response, it may be possible to utilize less antigen, e.g., about 1-5 .mu.g for a human subject. The appropriate amount of antigen to be used is dependent on the specific antigen and is well known in the art.

The exact effective amount necessary will vary from subject to subject, depending on the species, age and general condition of the subject, the severity of the condition being treated, the mode of administration, etc. Thus, it is not possible to specify an exact effective amount. However, the appropriate effective amount may be determined by one of ordinary skill in the art using only routine experimentation or prior knowledge in the vaccine art.

An “immunological response” to a composition or vaccine comprised of an antigen is the development in the host of a cellular- and/or antibody-mediated immune response to the composition or vaccine of interest. Usually, such a response consists, of the subject producing antibodies, B cells, helper T cells, suppressor T cells, NK cells, macrophages, granulocytes and/or cytotoxic T cells directed specifically to an antigen or antigens included in the composition or vaccine of interest.

Antigens and Vaccines

Synthetic antigens, including vaccines, may be prepared by chemically synthesizing peptides sharing antigenic determinants with proteins, for example, of HIV-1, rubella virus, RSV, Haemophilus influenzae type b, Bordetella pertussis and Streptococcus pneumoniae or other antigens. These peptides, lipid derivatives of such peptides as well as viral antigens or bacterial antigens, may be used either individually or combined as a cocktail, and formulated with the endogenous hormone adjuvant of the present invention, synthetic adjuvants, and/or mineral salts to provide an immunogenic composition. As contemplated herein, an antigen may be covalently bonded to a glycolipid analog to provide a discrete molecule which exhibits an enhanced adjuvanting effect on the antigen which is greater than the adjuvanting effect attainable in the absence of such covalent bonding. These compositions can be used to immunize mammals, for example, by the intramuscular or parenteral routes, or by delivery to mucosal surfaces using microparticles, capsules, liposomes and targeting molecules, such as toxins and antibodies.

An antigenic fraction of a pathogen can be produced by means of chemical or physical decomposition methods, followed, if desired, by separation of a fraction by means of chromatography, centrifugation and similar techniques. In general, low molecular components are then obtained which, although purified, may have low immunogenicity. Alternatively, antigens or haptens can be prepared by means of organic synthetic methods, or, in the case of, for example, polypeptides and proteins, by means of recombinant DNA methods.

Vaccines containing peptides are generally well known in the art, as exemplified by U.S. Pat. Nos. 4,601,903, 4,599,231; 4,599,230; and 4,596,792; all of which references are incorporated herein by reference. The use of peptides in vivo may first require their chemical modification since the peptides themselves may not have a sufficiently long serum and/or tissue half-life and/or sufficient immunogenicity. In addition, it may be advantageous to modify the peptides in order to impose a conformational restraint upon them. This might be useful, for example, to mimic a naturally-occurring conformation of the peptide in the context of the native protein in order to optimize the effector immune responses that are elicited.

The antigen or vaccine utilized in the present invention may be an antigen for a disease state or infectious agent, such as a virus or pathogen. An antigen or vaccine may comprise an antigen for a disease state, particularly that selected from the group consisting of smallpox, yellow fever, distemper, cholera, fowl pox, scarlet fever, diphtheria, tetanus, whooping cough, influenza,: rabies, mumps, measles, foot and mouth disease, poliomyelitis, viral hepatitis, influenza, diphtheria, tetanus, pertussis, measles, mumps, rubella, polio, pneumococcus, herpes, respiratory syncytial virus, haemophilus influenza type b, varicella-zoster virus or rabies.

Infectious agents include but are not limited to: viruses, bacteria, fungi, pathogenic, or non-pathogenic agents. Viruses, bacteria, and other pathogens include but are not limited to: avian encephalomyelitis virus, avian reovirus, avian paramyxovirus, avian influenza virus, avian adenovirus, fowl pox virus, avian coronavirus, avian rotavirus, chick anemia virus (agent), Salmonella spp. E. coli, Pasteurella spp., Bordetella spp., Eimeria spp., Histomonas spp., Trichomonas spp., avian encephalomyelitis virus, avian reovirus, avian paramyxovirus, avian influenza virus, avian adenovirus, fowl pox virus, avian coronavirus, avian rotavirus, chick anemia virus (agent), Salmonella spp. E coli, Pasteurella spp., Bordetella spp., Eimeria spp., Histomonas spp., Trichomonas spp., Poultry nematodes, cestodes, trematodes, poultry mites/lice, poultry protozoa.

Viruses of nonhuman primates are included but not limited to: Aotine herpesvirus 1, Aotine herpesvirus 3, Cercopithecine herpesvirus 1 (B virus, HV simiae), Cercopithecine herpesvirus 2 (SA8), Cercopithecine herpesvirus 3 (SA6), Cercopithecine herpesvirus 4 (SA15), Cercopithecine herpesvirus 5 (African green monkey cytomegalovirus), Cercopithecine herpesvirus 6 (Liverpool vervet monkey virus), Cercopithecine herpesvirus 7 (Patas monkey HV; MMV or PHV delta HV), Cercopithecine herpesvirus 8 (Rhesus monkey cytomegalovirus), Cercopithecine herpesvirus 9 (Medical Lake macaque LV simian varicella HV) , Cercopithecine herpesvirus 10 (Rhesus leukocyte assoc. LV strain 1T), Cercopithecine herpesvirus 12 (LV papio, baboon HV), Cercopithecine herpesvirus 13 (Herpesvirus cyclopis), Cercopithecine herpesvirus 14 (African green monkey EBV-like virus), Cercopithecine herpesvirus 15 (Rhesus EBV-like HV), Ateline herpesvirus 1 ( Spider monkey HV), Ateline herpesvirus 2 (HV ateles), Callitrichine herpesvirus (HV saguinus), Callitrichine herpesvirus (SSG, marmoset cytomegalovirus), Cebine herpesvirus 1 (Capuchin HV), Cebine herpesvirus 2 (Capuchin HV), Pongine herpesvirus 1 (Chimpanzee HV;pan HV), Pongine herpesvirus 2 (Orangutan HV), Pongine herpesvirus 3 (Gorilla HV), Saimiriine herpesvirus 1 (Marmoset HV, herpes T, HV), tamarinus, HV platyrrhinae, ( type Saimiriine herpesvirus 2) Squirrel monkey HV, and HV saimiri.

Viruses of mammals include but are not limited to: Bovine herpesvirus 1-5, Ovine herpesvirus 1-2, Alcelaphine herpesvirus 1, Parvovirus (including mice minute virus, Aleutian mink disease, bovine parvovirus, canine parvovirus, chicken parvovirus, feline panleukopenia, feline parvovirus, goose parvovirus, HB parvovirus, H-1 parvovirus, Kilham rat lapine parvovirus, mink enteritis) Erythrovirus (including adeno-associated type 1-5, bovine adeno-associated, canine adeno-associated, equine adeno-associated, ovine adeno-associated).

Viruses include but are not limited to: Cauliflower, Badnaviruses, Geminiviruses, Plant Reoviruses, Cryptoviruses, Rhabdoviridae, Tomato Spotted, Tenuiviruses, Tobacco, Potato Virus, Potyviridae, Closteroviruses, Turnip Yellow, Tomato Bushy, Luteoviruses, Sequiviridae, Tobacco, Cowpea, Tobacco, Pean Enation, Red Clover, Brome, Cucumber, Alfalfa, Barley, Beet Necrotic, and dsRNA.

Further viruses from the following family are included: Baculoviridae and Nudiviruses, Polydnaviridae, Ascoviridae, Nodaviridae Tetraviridae, Tetraviridae, Tombusviridae, Coronaviridae, Flaviviridae, Togaviridae, Bromoviridae, Barnaviridae, Totiviridae, Partitiviridae, Hypoviridae, Paramyxoviridae, Rhabdoviridae, Filoviridae, Orthomyxoviridae, Bunyavinrdae, Arenaviridae, Leviviridae, Picornaviridae, Sequiviridae, Comoviridae, Potyviridae, Calciviridae, Astroviridae, Nodaviridae, Inoviridae, Microviridae, Geminiviridae, Circoviridae, Parvoviridae, Haepadnaviridae, Retroviridae, Cystoviridae, Reoviridae, Birnaviridae, Myoviridae, Siphoviridae, Podoviridae, Tectiviridae, Corticoviridae, Plasmaviridae, Lipothrixviridae, Fuselloviridae, Poxviridae, African swine fever-like viruses, Iridoviridae, Phycodnaviridae, Baculoviridae, Herpesviridae, Adenoviridae, Papovaviridae, Polydnaviridae, Picornaviridae, Caliciviridae, Astroviridae, Togaviridae, Flaviviridae, Coronaviridae, Arterivirus, Paramyxoviridae, Rhabdoviridae, Filoviridae, Orthomyxoviridae, Bunyaviridae, Arenaviridae, Reoviridae, Birnaviridae, Retroviridae, Hepadnaviridae, Circoviridae, Parvoviridae, Papovaviridae, Adenoviridae, Herpesviridae, Poxviridae, Iridoviridae,

Viruses and other infectious agents, bacteria, or pathogens include but are not limited to: Marek's disease virus (fowl), Mink Enteritis virus, Minute virus of mice, Mouse hepatitis viruses, Mouse mammary tumor virus, Mouse poliomyelitis virus (Theile's virus) Mucosal disease virus (cattle), Myxoma virus, Nairobi sheep disease virus, Newcastle disease virus (fowl), orf virus (contagious pustular dermatitis virus), Parainfluenza virus 3, Parainfluenza virus 1 (Sendai virus), Peste-des-petits-ruminants virus (sheep and goats), Pneumonia virus of mice, Progressive penumonia virus of sheep, Psudocowpox virus (milker's nodule virus), Pseudorabies virus, Rabbit hemorrhagic disease virus, Rabies virus, Reoviruses 1-3, Rift Valley fever virus, Rinderpest virus, Rotaviruses of many species, Scrapie agent (sheep and goat), Sheeppox virus, Shope papillomavirus, Simian immunodeficiency viruses, Swine vesicular disease virus, Swinepox virus, Tick-borne encephalitis viruses, Transmissible gastroenteritis virus (swine), Turkey bluecomb virus, Venezuelan equine encephalitis virus, Vesicular exanthema virus (swine), Vesicular stomatitis virus, Wasting disease of deer and elk, Wesselsbron virus, Western equine encephalitis virus, African horsesickness viruses 1-9, African swine fever virus, Aleutian mink disease virus, Avian reticuloendotheliosis virus, Avian sarcoma and leukosis viruses, B virus (Cercopithecus herpesvirus), Berne virus (horses), Bluetongue viruses 1-25, Border disease virus (sheep), Borna disease virus (horses), Bovine enteroviruses 1-7, Bovine ephemeral fever virus, Bovine immunodeficiency virus, Bovine leukemia virus, Bovine mamillitis virus, Bovine papillomaviruses, Bovine papillomaviruses, Bovine papular stomatitis virus, Bovine respiratory syncytial virus, Bovine virus diarrhea virus, Breda virus (calves), Canine adenovirus 2, Canine distemper virus, Canine parvovirus, Caprine arthritis-encephalitis virus, Cowpox virus, Eastern equine encephalitis virus, Ebola virus, Ectromelia virus (mousepox virus), Encephalomyocarditis virus, Epizootic hemorrhagic disease viruses (deer), Equine abortion virus (EHV1), Equine adenoviruses, Equine arteritis virus, Equine coital exanthema virus (EHV3), Equine infectious anemia virus, Equine rhinopneumonitis virus (EHV4), Feline calicivirus, Feline immunodeficiency virus, Feline infectious peritoniitis virus, Feline panleukopenia virus, Feline sarcoma and leukemia viruses, Fibroma viruses of rabbits and hares and squirrels, Foot-and-mouse disease viruses, Fowipox virus, Hemagglutinating encephalomyelitis virus (swine), Hog cholera virus, Infectious bovine rhinotrachetitis virus, Infectious bronchitis virus (fowl), Infectious bursal disease virus (fowl), Infectious canine hepatitis virus, Infectious hematopoietic necrosis virus (fish), Infectious laryngotrachetis virus, infectious hematopoietic necrosis virus (fish), Influenza viruses of swine, horses, seals, and fowl, Japanese encephalitis virus, Lactic dehydrogenase virus (mice), Lymphocytic choriomeningitis virus, Maedi/visna virus (sheep), Marburg virus, Rocio virus, Ross River virus, Rubella virus, Russian spring-summer encephalitis virus, Sandfly fever-Naples virus, Sandfly fever-Sicilian virus, St. Louis encephalitis virus, SV 40 virus, Tahyna virus, Vaccinia virus, Varicella-zoster virus (human herpesvirus 3), Variola virus, Venezuelan equine encephalitis virus, Vesicular stomatitis viruses, West Nile virus, Eastern equine encephalitis virus, Yellow fever virus, Adenovirus 1-49, Astrovirus 1, 2, B virus (Cercopithecus herpesvirus), BK virus, Bunyamwera virus, California encephalitis virus, Central European encephalitis virus, Chikungunya virus, Colorado tick fever virus, Congo-Crimean hemorrhagic fever virus, Cowpox virus, Coxsacieviruses A 1-21 and A 24, Coxsackieviruses B 1-6, Creutzfeldt-Jakob disease agent, prions, Dengue viruses 1-4, Duvenhage virus, Eastern equine encephalitis virus, Ebola virus, Echoviruses 1-9 and 11-27 and 29-34, Enteroviruses 68-71, Epstein-Barr virus (human herpesvirus 4), Hantaan virus, Hepatitis A virus, Hepatitis B virus, Hepatitis C virus, Hepatitis delta virus, Hepatitis E virus, Herpes simplex viruses 1 and 2 (human herpesviruses 1 and 2), Human enteric coronavirus, Human enteric conoravirus, Human cytomeglovirus (human herpesvirus 5), Human herpesviruses 6A, 6B, and 7, Human immunodeficiency viruses 1 and 2 Human respiratory coronaviruses 229E and OC43, Human rotaviruses, Human T-lymphotropic viruses 1 and 2, Influenza viruses A and B, Japanese encephalitis virus, JC virus, Junin virus (Argentine hemorrhagic fever virus), Kuru agent, Kyasanur forest virus, La Crosse virus, Lassa virus, Lymphocytic choriomeningitis virus, Macuopo virus (Bolivian hemorrhagic fever virus), Marburg virus, Mayaro virus, Measles virus, Mokola virus, Molluscum contagiosum virus, Monkeypox virus, Muerto Canyon virus, Mumps virus, Murray Valley encephalitis virus, Norwalk virus (and related viruses), O'nyong-nyong virus, Omsk hemorrhagic fever virus, Orf virus (contagious pustular dermatitis virus), Oropouche virus, Papillomaviruses 1-60, Parainfluenza viruses 1 and 3, Parainfluenza viruses 2 and 4, Parvovirus B-19, Polioviruses 1-3, Pseudocowpox virus (milker's nodule virus), RA-1 virus, Rabies virus, Respiratory syncytial virus, Rhinoviruses 1-113, Rift Valley fever virus; Staphylococcal abscesses; Staphylococcal pneumonia; Staphylococcal bacteremia; Staphylococcal osteomyelitis; Staphylococcal; Influenza viruses A, B, and C; Parainfluenza viruses 1-4; Mumps virus; Adenoviruses; Reoviruses; Respiratory syncytial virus; Epstein-Barr virus; Rhinoviruses; Polioviruses; Colorado tick fever; Phlebotomus fever; Venezuelen equine encephalitis; Rift valley fever; Dengue fever; West Nile fever; Barmah Forest virus; Chikungunya disease; Mayaro virus disease; Ross river virus disease; Sindbis virus disease (Okelbo disease, Pogosta disease, Karelian fever); Eastern equine encephalitis, Western equine encephalitis; St. Louis encephalitis; Venezuelen equine encephalitis; California virus group, Japanese encephalitis, Powassan virus; Murray Valley encephalitis; Kyasanur Forest disease; Tick-borne encephalitis virus; Lymphocytic chotiomeningitis; Yellow fever; Dengue hemorrhagic fever; Kyasanur Forest disease; Omsk hemorrhagic fever; Crimean-Congo hemorrhagic fever; Hantaan virus; Seoul virus; Puumala virus; Machupo virus; Junin virus; Lassa fever; Marburg virus; Ebola virus; lasmodium spp; Trypanosoma spp; Microfilarie; Leishmania spp; naegleria Hartmannella Acanthamoeba group; Giardia lamblia, Strongyloides, Entamoeba histolytica, Schistosoma mansoni, Schistosoma japonicum; Entamoeba histolytica, Other amebas; Giardia lamblia; Cryptosporidium; Trichuris trichiura, Ascaris lumbricoides, Hookworm, Strongyloides, Tapeworm, Fluke; Enterobius vermicularis; Entamoeba histolytica; Paragonimus westermani; Entamoeba histolytica, Strongyloides, Echinococcus granulosus, Hookworm, Ascaris spp, Pneumocystis carinii; Pneumocystis carinii; Onchocerca volvulus; Leishmania spp, Entamoeba histolytica; Taenia solium; Trichomonas spp; Schistosoma haematobium, Cryphonectria parasitica, Giardia Lamblia, Chlorella and Saccharomyces cerevisiae.

Pathogens include but are not limited to: feline pathogen, canine pathogen, equine pathogen, bovine pathogen, avian pathogen, porcine pathogen, or human pathogen. Human pathogen includes but is not limited to: herpes simplex virus-1, herpes simplex virus-2, human cytomegalovirus, Epstein-Barr virus, Varicell-Zoster virus, human herpesvirus-6, human herpesvirus-7, human influenza, human immunodeficiency virus, rabies virus, measles virus, hepatitis B virus and hepatitis C virus. Furthermore, the antigenic polypeptide of a human pathogen may be associated with malaria or malignant tumor from the group consisting of Plasmodium falciparum, Bordetella.

Equine pathogen can derived from equine influenza virus or equine herpesvirus. Examples of such antigenic polypeptide are equine influenza virus type A/Alaska 91 neuraminidase, equine influenza virus type A/Prague 56 neuraminidase, equine influenza virus type A/Miami 63 neuraminidase, equine influenza virus type A/Kentucky 81 neuraminidaseequine herpesvirus type 1 glycoprotein B, and equine herpesvirus type 1 glycoprotein D.

Bovine pathogens include but are not limited to: bovine respiratory syncytial virus or bovine parainfluenza virus. The antigenic polypeptide of derived from bovine respiratory syncytial virus equine pathogen can derived from equine influenza virus is bovine respiratory syncytial virus attachment protein (BRSV G), bovine respiratory syncytial virus fusion protein (BRSV F), bovine respiratory syncytial virus nucleocapsid protein (BRSV N), bovine parainfluenza virus type 3 fusion protein, and the bovine parainfluenza virus type 3 hemagglutinin neuraminidase.

Therapeutic Compositions and Uses

The therapeutic uses of the endogenous hormone adjuvant or immunodulator of the present invention are contemplated as exemplified herein and further in view of the fact that certain endogenous stress hormones, particularly epinephrine and glucorticoids have been demonstrated to act as immunomodulators and activate the immune system, under doses and/or conditions that mimic the acute stress state. In as much as these are endogenous hormones, the untoward effects and limitations of previously known and utilized adjuvants, which are not natural or endogenous compounds, are avoided. In addition, this invention contemplates that natural or synthetic analogs of epinephrine or glucocorticoids would function similarly as immunomodulators, immune system activators and adjuvants.

The term “endogenous hormone adjuvant (EHA)”, “endogenous hormone immunodulator”, and any variants not specifically listed, may be used herein interchangeably, and as used throughout the present application and claims refer to hormone compositions, and extends to those described and identified herein, and the profile of activities set forth herein and in the claims. Accordingly, hormones or compounds displaying substantially equivalent or altered activity are likewise contemplated. These modifications may be deliberate, for example, such as modifications obtained through synthesis or site-directed mutagenesis, or may be accidental, such as those obtained through mutations in hosts that are producers of the hormones. Also, the term “endogenous hormone adjuvant (EHA)” is intended to include within its scope proteins specifically recited herein as well as all substantially homologous analogs and derivatives.

This invention provides a hormone adjuvant composition comprising an amount of endogenous epinephrine, endogenous glucocorticoids, or analogs thereof and combinations thereof In one embodiment the endogenous glucocorticoid is cortisol or corticosterone. In a further embodiment, the hormone adjuvant composition comprises an amount of an analog, particularly a synthetic analog, of epinephrine or of glucocorticoids, or combinations of such analogs. As contemplated herein, the hormone adjuvant may be artificially, synthetically, or commercially produced when in a formulation or pharmaceutical.

Analogs, including natural or synthetic analogs, of epinephrine and glucocorticoids for use in the present invention and compositions can be generated or isolated by methods well known in the art. A number of synthetic analogs of epinephrine and glucocorticoids are known and already identified, including but not limited to those provided in Goodman and Gilman, which reference is fully incorporated herein. (Gilman, A. G. et al, The Pharmacological Basis of Experimental Therapeutics, Pergamon, N.Y. (1990). Examples of synthetic cortisol analogs include, but are not limited to, prednisone, prednisolone, cortisone, and corticosterone. Examples of epinephrine or adrenaline analogs include, but are not limited to, methoxamine, clonidine, p-aminoclonidine Hcl, guanabenz acetate, p-iodoclonidine Hcl, UK 14.304, Xylazine Hcl, and isoproterenol. Any analog would be selected and suitable on the basis of its similarity in structure and function (e.g. receptor recognition and affinity, concentration of effective dose, half-life, etc.). For instance, as shown in the Examples herein, the synthetic analog dexamethasone, tested by the methods described herein, is not a suitable analog, failing to demonstrate adjuvant activity and showing immune-suppressing activity. As noted herein, particulary in Example 2, dexamethasone displays significant differences from endogenous glucocorticoids in that it (a) does not bind corticosteroid binding globulin, a plasma protein which binds a large proportion of circulating corticosterone; (b) has a significantly longer half-life than corticosterone; (c) has a higher affinity for glucocorticoid receptors; and (d) is significantly more efficient than corticosterone at activating glucocorticoid receptors in vivo.

This invention also provides a therapeutic composition, comprising a mixture of a therapeutically effective antigen or vaccine; and a hormone adjuvant composition of endogenous epinephrine, endogenous glucocorticoids, or analogs thereof and combinations thereof, and a suitable carrier or diluent. In one embodiment the therapeutic composition comprises at least one antigenic agent selected from the group consisting of (A) viruses, pathogens, bacteria, mycoplasmas, fungi, protozoa and other infectious agents; (B) fragments, extracts, subunits, metabolites, and recombinant constructs of (A); (C) fragments, subunits, metabolites, and recombinant constructs of mammalian proteins or glycoproteins, (D) tumor-specific antigens; (E) pathogenic organisms and non-pathogenic organisms; and (F) combinations thereof

The hormone adjuvant also may be used to enhance cellular, humoral or antibody-mediated immunity. In enhancing cellular immunity, the endogenous hormone adjuvant may be used during the sensitization phase or the challenge phase of immunization, or at both phases, and may be used alone or in combination with previously known adjuvants (e.g. Freund's adjuvant, BCG or mineral salts).

The invention further provides a pharmaceutical composition, comprising an endogenous hormone adjuvant or immunomodulator composition of endogenous epinephrine, endogenous glucocorticoids, or analogs thereof and combinations thereof and a suitable carrier or diluent.

Thus, this invention further contemplates the hormone adjuvant being utilized to stimulate the immune system or an immune response in the absence of a vaccine, for example, as a prelude to surgery, to combat disease, or during an infection. The hormone adjuvant may be injected to induce movements of immune cells, for example, leukocytes, out of some compartments (such as blood or spleen) into other such as skin, lymph nodes, and bone marrow. This is useful, for example, in cases of cancer therapy where a specific region is being irradiated and the cocktail could be used to move cells out of that region and into safer areas.

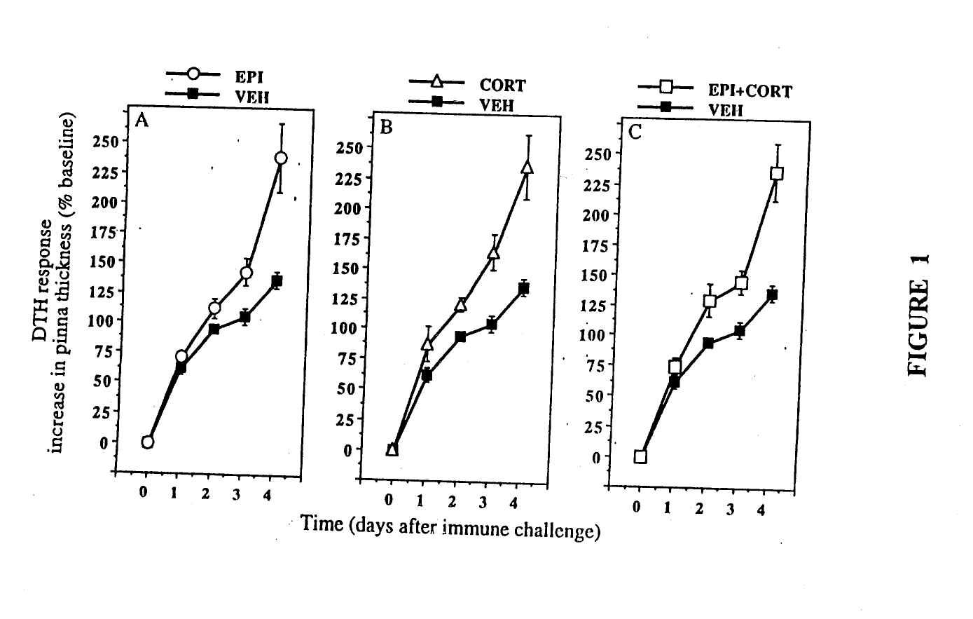

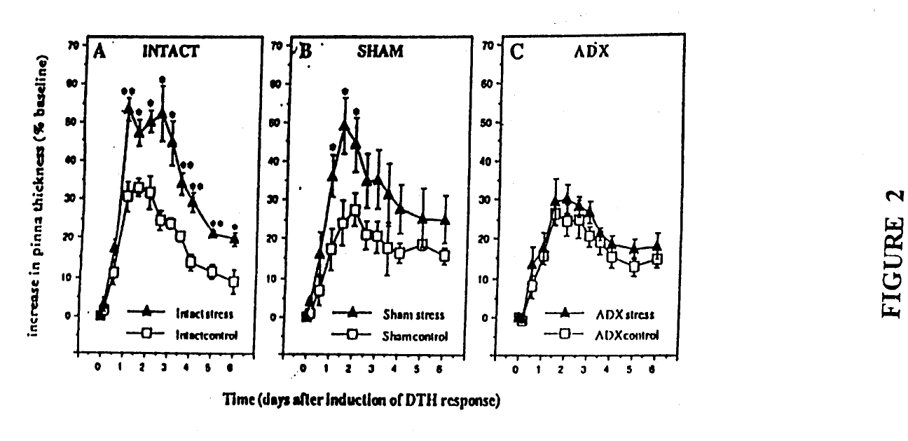

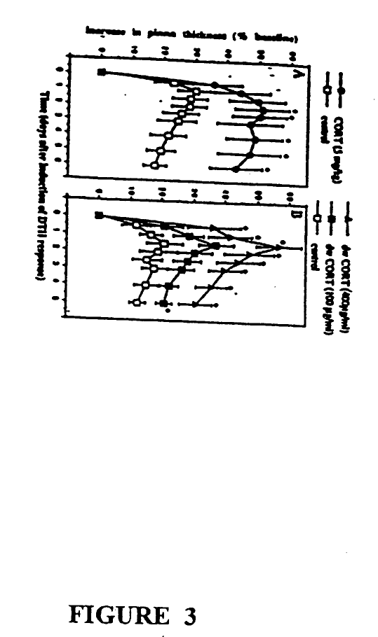

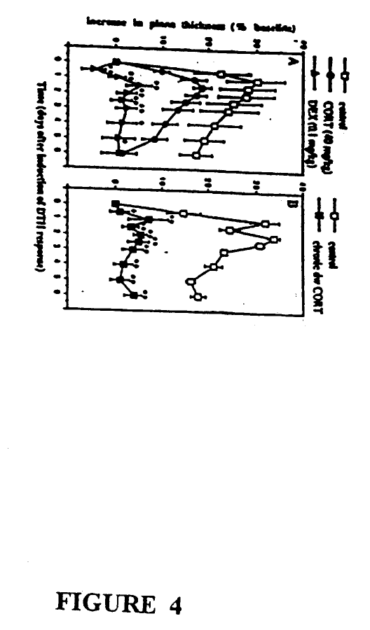

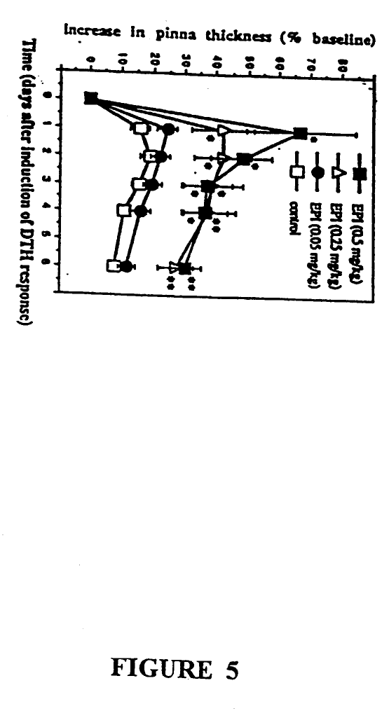

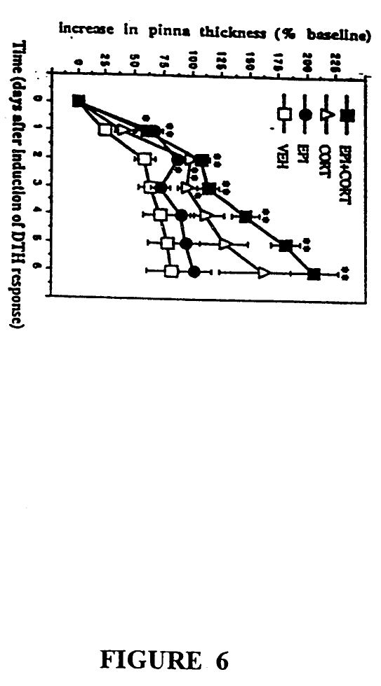

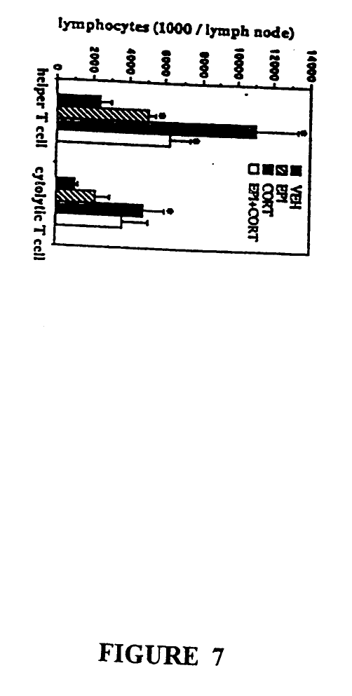

The endogenous hormone adjuvant may be administered in conjunction with vaccines during the immunization phase. As demonstrated herein, stress hormones, particularly epinephrine and the glucocorticoid corticosterone, administered during the sensitization phase of a DTH response, enhance the immune reaction observed during the challenge or recall phase of DTH. The DTH response of two groups of animals was compared (FIG. 1). One group of animals, the control group, was injected with vehicle (VEH, control for injection) at the time of immunization with a novel antigen (oxazalone, OXA). The second group was injected with a “hormone cocktail” or EHA consisting of a low-dose formulation of epinephrine and corticosterone (epinephrine (0.5 mg/kg)+corticosterone (5 mg/kg)). The strength of a cell-mediated immune response or DTH reaction mounted against OXA was examined six days after the initial immunization and EHA administration. This was accomplished by challenging the skin (dorsal aspect of ear) with a low concentration of OXA. Compared to VEH treated controls, the DTH response of hormone injected animals occurred at a faster rate, attained a higher peak, and remained significantly higher for several days after challenge. These studies show that EHA administration during immunization with OXA significantly enhances a subsequent immune response mounted following re-exposure of the animals to OXA.

The advantages of using ERA are especially relevant in light of the numerous adverse reactions to agents such as aluminum compounds (Fiejka, M and Aleksandrowicz, J., Roczniki Pahstwowego Zakladu Higieny 44: 73 (1993)), bacterial extracts (Yamanaka, et al., J. Vet. Medical Science 54:685 (1992)), viral extracts (Yamanaka et al., J. Vet. Medical Science 56:185 (1994), and oil emulsions (Yamanaka et al., J. Vet. Medical Science 56:185 (1994), which are currently used as adjuvants to enhance immune function in conjunction with vaccine administration and other medical treatments (Leenaars, P. P. et al., Vet. Immunol & Immunopath. 48:123 (1995)). These adverse effects include pathological reactions at the site of injection (Yamanaka, et al., J. Vet. Medical Science 56:185 (1994)), anaphylactic reactions (Marcos, C. et al., Allergy 46:235 (1991)), arthritic reactions (Kohasi, O. et al., Internatl. Archives Allergy & Applied Immunol. 53:537 (1977), muscle damage (Goto, N. and Akana, K., Microbiol. & Immunol 26:1121 (1982), and tumor formation (Suster, S. M. et al., Oncology 44:279 (1987)). Since the EHA would consist of substances already manufactured by the body, and given in doses similar to those secreted by the body, its adverse effects are expected to be minimal. Since these hormones are endogenously produced, the body is equipped to “handle” them, both, in terms of being receptive to their biologic actions, and in terms of rapidly metabolizing or clearing them from the body once they have performed their function. Moreover, the doses of hormones used would be in the low to moderate range such as those attained naturally during the course of a day. These factors significantly reduce potential side effects which are often induced by non-endogenous agents which are more resistant to removal from the body. Similarly, analogs of endogenous epinephrine or endogenous glucocorticoids, including synthetic analogs, which retain activity (including receptor recognition, dose range of effectiveness, etc) and/or structure similar to endogenous epinephrine or endogenous glucocorticoids would have the same advantages and reduced side effects.

An important distinction between the well-known clinical applications of these hormones, and the application proposed here, is the concentration which would be administered, and the fact that, in a particular embodiment, cortisol and epinephrine would be administered in combination. For example, high doses of synthetic analogs of cortisol are widely used for immunosuppression during pro-inflammatory and autoimmune disorders (Schleimer, R. P. et al., Anti-Inflammatory Steriod Action, Academic Press Inc., San Diego, Calif. (1989); Haynes, R. C. J. in The Pharmacological Basis of Experimental Therapeuities, Gilman, et al, eds. Pergamon, N.Y., pp. 1431-1462 (1990)). In contrast, EHA would consist of significantly lower doses of the natural hormones cortisol and epinephrine, or analogs thereof, which have been shown to significantly enhance immune function. Delayed type hypersensitvity (DTH) reactions are antigen-specific, cell-mediated immune responses which, depending on the antigen, mediate beneficial (resistance to viruses, bacteria, and fungi) or harmful (allergic dermatitis, autoimmunity) aspects of immune function. Contrary to the notion that stress suppresses immunity, it is shown herein, that short duration stressors significantly enhance skin DTH and that a stress-induced trafficking of leukocytes to the skin may mediate this immuno-enhancement. Adrenalectomy, which eliminates the glucocorticoid and epinephrine stress response, eliminated the stress-induced enhancement of skin DTH. Low dose corticosterone or epinephrine administration significantly enhanced skin DTH. In contrast, high dose corticosterone, chronic corticosterone, or low dose dexamethasone administration, significantly suppressed skin DTH. These results suggest a novel role for adrenal stress hormones as endogenous immuno-enhancing agents. They also show that stress hormones released during a circumscribed or acute stress response may help prepare the immune system for potential challenges (e.g. wounding or infection) for which stress perception by the brain may serve as an early warning signal. As such as contemplated, this invention provides a method of promoting an acute stress induced enhancement of immunity and a method of enhancing antigen processing, cytokine production and antibody production by administering to the subject the endogenous hormone adjuvant composition.

This invention provides a method of stimulating or enhancing an antigen-specific cell-mediated immune response which comprises administering to a subject an amount of an immunomodulator or antigen as a vaccine and a low dose of the hormone adjuvant composition comprising an amount of endogenous epinephrine, endogenous glucocorticoids, or analogs thereof, and combinations thereof. In one embodiment, the hormone adjuvant composition is administered prior to vaccination. In another embodiment the hormone adjuvant composition is administered contemporaneously with vaccination. In another embodiment the hormone adjuvant composition is administered in a vaccine. In a further embodiment, the hormone adjuvant is administered in a booster vaccine to enhance immune response. Further, the hormone adjuvant of the present invention may be administered in combination with previously known adjuvants (e.g. Freund's adjuvant, BCG, and mineral salts). As contemplated herein, a subject may be vaccinated for any infectious agent.

This invention provides such a “hormone cocktail” or EHA administration in conjunction with vaccination (e.g. with hepatitis, tetanus, or BCG vaccines) in order to enhance the efficacy of the vaccine and hence confer protection on the subject, the subject to be subsequently exposed to, for example, hepatitis, tetanus, or tuberculosis. This invention provides a method for conferring protection against an infectious agent which comprises administering to a subject an amount of an immunomodulator as a vaccine and a low dose of the endogenous hormone adjuvant composition in an amount of endogenous epinephrine, endogenous glucocorticoids, or analogs thereof, and combinations thereof, and a suitable carrier or diluent.

An important application of EHA would be to naturally enhance an immune response at the time of vaccine administration. Such immuno-enhancement could have two important benefits: First, vaccines delivered in conjunction with EHA could result in a stronger, and longer-lasting immunologic memory against the infectious agent for which the vaccine is intended. This would confer protection against the infectious agent for longer periods of time thus reducing the number of “booster shots” necessary to maintain immunity. For example, Tetanus vaccines need frequent boosters to maintain immunity, and this booster frequency could be lowered by administering EHA in conjunction with tetanus vaccine. Second, immuno-enhancement at the time of vaccine administration could make it possible to reduce the concentration of antigens which constitute the vaccine. This is beneficial in the case of antigens which are known to induce discomfort, fever, or illness following administration. Administering lower doses of such antigens would reduce or eliminate the discomfort or illness following vaccine administration. Reduction of adverse effects could be especially beneficial for infants and the elderly in whom post-vaccination discomfort and illness may be more severe.

This invention provides a method of treating a subject with an infectious agent or cancer comprising administering to a subject an amount of the hormone adjuvant composition of the present invention as an immunomodulator, comprising a low dose of endogenous epinephrine, endogenous glucocorticoids, or analogs thereof, and combinations thereof, and a suitable carrier or diluent. In particular, a subject having cancer may be treated with the endogenous hormone adjuvant composition. Such cancers include but are not limited to: melanoma; lymphoma; leukemia; and prostate, colorectal, pancreatic, breast, brain, or gastric carcinoma. Examples of tumors include but are not limited to: include sarcomas and carcinomas such as, but not limited to: fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's sarcoma, leiomyosarcoma, rhabdomyosarcoma, colon carcinoma, pancreatic cancer, breast cancer, ovarian cancer, prostate cancer, squamous cell carcinoma, benign prostate hyperplasia, prostate, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, germ tumor, non-small cell lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, and retinoblastoma. In a preferred embodiment the tumor cell is a urothelial cell.

For example, a modulated regimen of EHA administration would be used to rev up the immune response of a patient bearing a localized infection. Similarly, EHAs would he used in conjunction with chemotherapy, radiation therapy or immunotherapy for cancer. In this case, the immuno-enhancement may be beneficial not only for fighting the cancer, but also for protecting the patient against opportunistic infections which occur as a result of a weakened immune system following chemotherapy or radiation therapy.

Further the subject may be treated with the endogenous hormone adjuvants in combination with chemotherapeutic, chemopreventive, or radiation therapy. It is contemplated by this invention that endogenous hormone adjuvant composition could be used in conjunction with chemo- or radiotherapeutic intervention. In another embodiment, treatment with the adjuvant composition may precede or follow the DNA damaging agent treatment by intervals ranging from minutes to weeks. Protocols and methods are known to those skilled in the art. DNA damaging agents or factors are known to those skilled in the art and means any chemical compound or treatment method that induces DNA damage when applied to a cell. Such agents and factors include radiation and waves that induce DNA damage such as, gamma -irradiation, X-rays, UV-irradiation, microwaves, electronic emissions, and the like. A variety of chemical compounds, also described as “chemotherapeutic agents”, function to induce DNA damage, all of which are intended to be of use in the combined treatment methods disclosed herein. Chemotherapeutic agents contemplated to be of use, include, e.g., adriamycin, 5-fluorouracil (5FU), etoposide (VP-16), camptothecin, actinomycin-D, mitomycin C, cisplatin (CDDP) and even hydrogen peroxide. Combinations of one or more DNA damaging agents may be used with the EHA, whether radiation-based or actual compounds, such as the use of X-rays with cisplatin or the use of cisplatin with etoposide. Other neoplastic or toxic agents include but are not limited: 5-fluorouracil, methotrexate and adriamycin which may be linked in each case to, for example, a cephalosporin (see WO-A94 01 137 and EP-A-0 382 411) or cephalosporin mustards (see EP-A-O 484 870).

In another embodiment one may irradiate the localized tumor site with DNA damaging radiation such as X-rays, UV-light, gamma -rays or even microwaves. Alternatively, the tumor cells may be contacted with the DNA damaging agent by administering to the subject a therapeutically effective amount of a pharmaceutical composition comprising a DNA damaging compound such as, adriamycin, 5-fluorouracil, etoposide, camptothecin, actinomycin-D, mitomycin C, or more preferably, cisplatin.

EHA provides a unique value for the individual since unlike many pharmacologic treatments, an EHA would harness the individual's natural defense system to fight disease (infection or cancer) or maintain health (vaccination). This would ensure that no undue load was placed on the other physiologic systems of the body while immune function is enhanced. Moreover, as discussed below, the probability of experiencing averse effects while using EHA would be low. This would be an advantage over currently used non-endogenous adjuvants which often have significant adverse effects. Moreover, since epinephrine and cortisol are not expensive to manufacture, EHAs would provide an economical approach to immuno-enhancement.

It is preferred that the EHA composition utilizes an injected or otherwise administered quantity which produces levels of glucocorticoid or epinephrine which would be observed under natural stress conditions.. For example, Rats: Corticosterone (1-5 mg/kg), Epinephrine (0.2-0.5 mg/kg). (Injections are ip or sub cut). Humans: Cortisol (0.2-0.5 mg/kg body weight), Epinephrine (2-5 microgm/kg). (Injections may be subcutaneous or intramuscular).

As indicated above, the present invention, in one embodiment, provides adjuvant mixtures useful for formulating immunogenic compositions, suitable to be used as, for example, vaccines. The immunogenic composition elicits an immune response by the host to which it is administered including the production of antibodies and immune cells by the host. The immunogenic compositions include at least one antigen in one embodiment. This antigen may be an inactivated pathogen or an antigenic fraction of a pathogen. The pathogen may be, for example, a virus, a bacterium or a parasite. The pathogen may be inactivated by a chemical agent, such as formaldehyde, glutaraldehyde, beta-propiolactone, ethyleneimine and derivatives, or other compounds. The pathogen may also be inactivated by a physical agent, such as UV radiation, gamma radiation, “heat shock” and X-ray radiation.

The adjuvant compositions may be prepared as injectables, as liquid solutions or emulsions. The antigens and immunogenic compositions may be mixed with physiologically acceptable carriers which are compatible therewith. These may include water, saline, dextrose, glycerol, ethanol and combinations thereof The vaccine may further contain auxiliary substances, such as wetting or emulsifying agents or pH buffering agents, to further enhance their effectiveness. Vaccines may-be administered by injection subcutaneously or intramuscularly.

Alternatively, the immunogenic compositions formed according to the present invention, may be formulated and delivered in a manner to evoke an immune response at mucosal surfaces. Thus, the immunogenic composition may be administered to mucosal surfaces by, for example, the nasal or oral (intragastric) routes. Alternatively, other modes of administration including suppositories may be desirable. For suppositories, binders and carriers may include, for example, polyalkylene glycols and triglycerides. Oral formulations may include normally employed incipients, such as pharmaceutical grades of saccharine, cellulose and magnesium carbonate.

These compositions may take the form of solutions, suspensions, tablets, pills, capsules, sustained release formulations or powders and contain 1 to 95% of the immunogenic compositions of the present invention. The immunogenic compositions are administered in a manner compatible with the dosage formulation, and in such amount as to be therapeutically effective, protective and immunogenic. The quantity to be administered depends on the subject to the immunized, including, for example, the capacity of the subject's immune system to synthesize antibodies, and if needed, to produce a cell-mediated, humoral or antibody-mediated immune response. Precise amounts of antigen and immunogenic composition to be administered depend on the judgement of the practitioner. However, suitable dosage ranges are readily determinable by those skilled in the art and may be of the order of micrograms to milligrams. Suitable regimes for initial administration and booster doses are also variable, but may include an initial administration followed by subsequent administrations. The dosage of the vaccine may also depend on the route of administration and will vary according to the size of the host.

The concentration of antigen in an immunogenic composition according to the invention is in general 1 to 95%. A vaccine which contains antigenic material of only one pathogen is a monovalent vaccine. Vaccines which contain antigenic material of several pathogens are combined vaccines and also belong to the present invention. Such combined vaccines contain, for example, material from various pathogens or from various strains of the same pathogen, or from combinations of various pathogens.

As used herein, “pharmaceutical composition” could mean therapeutically effective amounts of the endogenous hormone adjuvant composition of the invention together with suitable diluents, preservatives, solubilizers, emulsifiers, adjuvant and/or carriers. A “therapeutically effective amount” as used herein refers to that amount which provides a therapeutic effect for a given condition and administration regimen. Such compositions are liquids or lyophilized or otherwise dried formulations and include diluents of various buffer content (e.g., Tris-HCl., acetate, phosphate), pH and ionic strength, additives such as albumin or gelatin to prevent absorption to surfaces, detergents (e.g., Tween 20, Tween 80, Pluronic F68, bile acid salts). solubilizing agents (e.g., glycerol, polyethylene glycerol), anti-oxidants (e.g., ascorbic acid, sodium metabisulfite), preservatives (e.g., Thimerosal, benzyl alcohol, parabens), bulking substances or tonicity modifiers (e.g., lactose, mannitol), covalent attachment of polymers such as polyethylene glycol to the protein, complexation with metal ions, or incorporation of the material into or onto particulate preparations of polymeric compounds such as polylactic acid, polglycolic acid, hydrogels, etc, or onto liposomes, microemulsions, micelles, unilamellar or multilamellar vesicles, erythrocyte ghosts, or spheroplasts. Such compositions will influence the physical state, solubility, stability, rate of in vivo release, and rate of in vivo clearance. Controlled or sustained release compositions include formulation in lipophilic depots (e.g., fatty acids, waxes, oils). Also comprehended by the invention are particulate compositions coated with polymers (e.g., poloxamers or poloxamines). Other embodiments of the compositions of the invention incorporate particulate forms protective coatings, protease inhibitors or permeation enhancers for various routes of administration, including parenteral, pulmonary, nasal and oral.

The modes of administration may comprise the use of any suitable means and/or methods for delivering the adjuvant or adjuvant-containing vaccine to a corporeal locus of the host animal where the adjuvant and associated antigens are immumostimulatively effective. Delivery modes may include, without limitation, parenteral administration methods, such as paracancerally, transmucosally, transdermally, intramuscularly, intravenously, intradermally, subcutaneously, intraperitonealy, intraventricularly, intracranially and intratumorally.

Further, as used herein “pharmaceutically acceptable carrier” are well known to those skilled in the art and include, but are not limited to, 0.01-0.1M and preferably 0.05M phosphate buffer or 0.8% saline. Additionally, such pharmaceutically acceptable carriers may be aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's or fixed oils. Intravenous vehicles include fluid and nutrient replenishers, electrolyte replenishers such as those based on Ringer's dextrose, and the like. Preservatives and other additives may also be present, such as, for example, antimicrobials, antioxidants, collating agents, inert gases and the like.

Controlled or sustained release compositions include formulation in lipophilic depots (e.g. fatty acids, waxes, oils). Also comprehended by the invention are particulate compositions coated with polymers (e.g. poloxamers or poloxamines) and the compound coupled to antibodies directed against tissue-specific receptors, ligands or antigens or coupled to ligands of tissue-specific receptors Other embodiments of the compositions of the invention incorporate particulate forms protective coatings, protease inhibitors or permeation enhancers for various routes of administration, including parenteral, pulmonary, nasal and oral.

When administered, compounds are often cleared rapidly from mucosal surfaces or the circulation and may therefore elicit relatively short-lived pharmacological activity. Consequently, frequent administrations of relatively large doses of bioactive compounds may by required to sustain therapeutic efficacy. Compounds modified by the covalent attachment of water-soluble polymers such as polyethylene glycol, copolymers of polyethylene glycol and polypropylene glycol, carboxymethyl cellulose, dextran, polyvinyl alcohol, polyvinylpyrrolidone or polyproline are known to exhibit substantially longer half-lives in blood following intravenous injection than do the corresponding unmodified compounds (Abuchowski et al. in Enzymesa as Drugs, Holcerberg and Roberts eds., pp. 367-383 (1981); Katre et al., Proc. Natl. Acad. Sci. USA 84(6): 1487-91 (1987)). Such modifications may also increase the compound's solubility in aqueous solution, eliminate aggregation, enhance the physical and chemical stability of the compound, and greatly reduce the immunogenicity and reactivity of the compound. As a result, the desired in vivo biological activity may be achieved by the administration of such polymer-compound abducts less frequently or in lower doses than with the unmodified compound.

The preparation of therapeutic compositions which contain an active component is well understood in the art. Typically, such compositions are prepared as an aerosol of the polypeptide delivered to the nasopharynx or as injectables, either as liquid solutions or suspensions, however, solid forms suitable for solution in, or suspension in, liquid prior to injection can also be prepared. The preparation can also be emulsified. The active therapeutic ingredient is often mixed with excipients which are pharmaceutically acceptable and compatible with the active ingredient. Suitable excipients are, for example, water, saline, dextrose, glycerol, ethanol, or the like and combinations thereof In addition, if desired, the composition can contain minor amounts of auxiliary substances such as wetting or emulsifying agents, pH buffering agents which enhance the effectiveness of the active ingredient.

An active component can be formulated into the therapeutic composition as neutralized pharmaceutically acceptable salt forms. Pharmaceutically acceptable salts include the acid addition salts (formed with the free amino groups of the polypeptide or antibody molecule) and which are formed with inorganic acids such as, for example, hydrochloric or phosphoric acids, or such organic acids as acetic, oxalic, tartaric, mandelic, and the like. Salts formed from the free carboxyl groups can also be derived from inorganic bases such as, for example, sodium, potassium, ammonium, calcium, or ferric hydroxides, and such organic bases as isopropylamine, trimethylamine, 2-ethylamino ethanol, histidine, procaine, and the like.

In yet another embodiment, the therapeutic compound can be delivered in a controlled release system. For example, the polypeptide may be administered using intravenous infusion, an implantable osmotic pump, a transdermal patch, liposomes, or other modes of administration. In one embodiment, a pump may be used (see Langer, supra; Sefton, CRC Crit. Ref Biomed. Eng. 14:201 (1987), Buchwald et al., Surgery 88:507 (1980); Saudek et al., N. Engl. J. Med. 321:574 (1989). In another embodiment, polymeric materials can be used (see Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, N.Y. (1984); Ranger and Peppas, J. Macromol. Sci. Rev. Macromol. Chem. 23:61 (1983); see also Levy et al., Science 228:190 (1985); During et al., Ann. Neurol. 25:351 (1989); Howard et al., J. Neurosurg. 71:105 (1989). In yet another embodiment, a controlled release system can be placed in proximity of the therapeutic target, i.e., the brain, thus requiring only a fraction of the systemic dose (see, e.g., Goodson, in Medical Applications of Controlled Release, supra, vol. 2, pp. 115-138 (1984). Preferably, a controlled release device is introduced into a subject in proximity of the site of inappropriate immune activation or a tumor. Other controlled release systems are discussed in the review by Langer (Science 249:1527-1533 (1990).

As can be readily appreciated by one of ordinary skill in the art, the methods and pharmaceutical compositions of the present invention are particularly suited to administration to a mammal, preferable a human subject.

In the therapeutic methods and compositions of the invention, a therapeutically effective dosage of the active component is provided. A therapeutically effective dosage can be determined by the ordinary skilled medical worker based on patient characteristics (age, weight, sex, condition, complications, other diseases, etc.), as is well known in the art. Furthermore, as further routine studies are conducted, more specific information will emerge regarding appropriate dosage levels for treatment of various conditions in various patients, and the ordinary skilled worker, considering the therapeutic context, age and general health of the recipient, is able to ascertain proper dosing. Generally, for intravenous injection or infusion, dosage may be lower than for intraperitoneal, intramuscular, or other route of administration. The dosing schedule may vary, depending on the circulation half-life, and the formulation used. The compositions are administered in a manner compatible with the dosage formulation in the therapeutically effective amount. Precise amounts of active ingredient required to be administered depend on the judgment of the practitioner and are peculiar to each individual. Suitable regimes for initial administration and booster shots are also variable, but are typified by an initial administration followed by repeated doses at one or more hour intervals by a subsequent injection or other administration. Alternatively, continuous intravenous infusion sufficient to maintain concentrations of ten nanomolar to ten micromolar in the blood are contemplated.

The following examples are provided to describe and illustrate the present invention. As such, they should not be construed to limit the scope of the invention. Those in the art will well appreciate that many other embodiments also fall within the scope of the invention, as it is described hereinabove and in the claims.

### EXPERIMENTAL DETAILS SECTION

### EXAMPLE 1

Endogenous Hormone Adjuvant

Materials & Methods:

Young adult, male Sprague Dawley rats (150-300 g) (Harlan Sprague Dawley, Indianapolis, Ind.) were housed in plastic cages in the accredited (American Association of Accreditation of Laboratory Animal Care) animal facilities of The Rockefeller University (New York, N.Y.). The animal room was maintained on a 12 h light-dark cycle (lights on 7 am). Animals were given rat chow and water ad libitum.

Adrenalectomy: Bilateral adrenalectomy (ADX) was performed using standard aseptic surgical techniques on animals fully anesthetized with the inhalant, methoxyflurane (Metofane; Pitman-Moore, Washington Crossing, N.J.). ADX animals were maintained on a low “normalizing” dose of corticosterone (20 ug/ml) administered through drinking fluid (animals were given two bottles, one with water and one with 3% saline). This was necessary for restoring important permissive functions of corticosterone which are lost following ADX (18-20). Corticosterone replacement normalizes basal levels of adrenocorticotropic hormone (ACTH), blood leukocyte numbers, and catecholamine hormones (21), all of which are abnormally high in ADX animals. Unlike constant replacement (via pellets or osmotic pumps), drinking water corticosterone also facilitates the normal termination of a stress-induced ACTH response (22, 23), and simulates the circadian corticosterone rhythm as animals drink at the beginning of the active period (16).

Hormone administration: Vehicle or hormone were administered via intraperitoneal (ip) injection. Corticosterone (Sigma, St. Louis, Mo.) was dissolved in an aqueous solution of 2-Hydroxypropyl-b-cyclodextrin (30%, (HBC) RBI, Natick, Mass.). Epinephrine (RBI, Natick, Mass.) was dissolved in sterile water. The concentration of corticosterone injected was (5 mg/ml) with the injection quantity adjusted (depending on body weight) to attain a final dose of 5 mg/kg. The concentration of epinephrine one injected was (0.5 mg/ml) with the injection quantity adjusted to attain a final dose of 0.5 mg/kg. Hormones were injected two hours before the sensitization step described below.

Induction of delayed type hypersensitivity (DTH): DTH was induced by challenging the pinnae of previously sensitized rats with oxazalone (OXA, Sigma, St. Louis, Mo.). On day 0 (start day) of the experiment, animals were anesthetized with the inhalant, methoxyflurane (Metofane; Pitman-Moore, Washington Crossing, N.J.). No anesthesia was used subsequently. An area of approximately 3×4 cm was shaved on the dorsum. The thickness of both pinnae was recorded using a constant-loading dial micrometer (Mitutoyo, Japan). On days 1 and 2 of sensitization, one hundred microliters of OXA (1.5% (w/v) in ethanol) were applied to the shaved dorsum. The specified hormone solutions were administered to each animal via ip injection 2 hours before the sensitization.

On day 5, baseline pinna thickness was measured. On day 6, after stress or hormone administration, the dorsal surface of the right pinnae of all animals was challenged with 50 ul of OXA (0.75% in ethanol). Left pinnae were treated with vehicle. Pinna thickness was measured at the times shown with all measurements being made on same relative region of the pinna. Vehicle treated (left) pinnae showed no significant change in thickness.

The immune reaction induced using the above procedure, is characterized by swelling at the site of challenge, and by an infiltration of monocytes/macrophages and lymphocytes into the epidermis and dermis (29-31). A positive correlation between the intensity of the immune reaction and the increase in pinna thickness has been reported (32, 33). This model for skin DTH reactions has been widely used to monitor cell-mediated immune responses in vivo (30, 34).

Data analysis and statistics: For all experiments, repeated measures were made on each animal. Significant differences between timepoints were analyzed using the Student's t-test as a test for significant differences between means. Means that differed significantly are indicated by symbols that are defined in the figure legends. Data are expressed as mean +SEM in all figures. A computer statistics package was used for statistical analyses (SYSTAT v5.2.1, Systat Inc., Evanston, Ill.).

Source of hormone: The concentration and timing of each hormone administered is listed in the text and figures. Vehicle or hormone were administered via intraperitoneal (ip) injection. Corticosterone and dexamethasone (Sigma, St. Louis, Mo.) were dissolved in an aqueous solution of 2-Hydroxypropyl-b-cyclodextrin (30%, (HBC) RBI, Natick, Mass.). Epinephrine (RBI, Natick, Mass.) was dissolved in sterile water.

Results:

Adjuvants, or immuno-enhancing agents, are often administered in conjunction with vaccines during the immunization phase. These agents enhance the efficacy of the vaccine. As demonstrated herein, stress hormones, epinephrine and/or corticosterone, administered during the sensitization phase of a DTH response, enhance the immune reaction observed during the challenge or recall phase of DTH.

As shown in FIGS. 1A-C, the DTH response of four groups of animals is compared. One group of animals, the control group, was injected with vehicle (VEH) at the time of immunization with antigen (1.5% Oxazolone, OXA) (A, B, C). A second group was injected with epinephrine (0.5 mg/kg) (A) at the time of immunization, a third group with corticosterone (5 mg/kg) (B) at the time of immunization, and a fourth group with a “hormone cocktail” consisting of a low-dose formulation of epinephrine and corticosterone (epinephrine (0.5 mg/kg)+corticosterone (5 mg/kg))(C). The strength of a cell-mediated immune response or DTH reaction mounted against OXA was examined six days after immunization and hormone administration by challenging the skin (dorsal aspect of ear) with a low concentration of OXA.

Compared to VEH treated controls, the DTH response of all the hormone injected animals occurred at a faster rate, attained a higher peak, and remained significantly higher for several days after challenge. These studies show that EHA administration during immunization with OXA significantly enhances a subsequent immune response mounted following re-exposure (or challenge) of the animals to OXA.

### EXAMPLE 2

Enhancing Effects of Stress Hormones on Skin Immune Function

Materials and Methods:

Young adult, male Sprague Dawley rats (150-300 g) (Harlan Sprague Dawley, Indianapolis, Ind.) were housed in plastic cages in the accredited (American Association of Accreditation of Laboratory Animal Care) animal facilities of The Rockefeller University (New York, N.Y.). The animal room was maintained on a 12 h light-dark cycle (lights on 7 am). Animals were given rat chow and water ad libitum.

Bilateral adrenalectomy (ADX) was performed using standard aseptic surgical techniques on animals fully anesthetized with the inhalant, methoxyflurane (Metofane; Pitman-Moore, Washington Crossing, N.J.). Sham adrenalectomized (SHAM) animals went through exactly the same procedure as ADX animals except that their adrenals were not removed. ADX animals were maintained on a low normalizing dose of corticosterone (20 μg/ml) administered through drinking choice of water or 3% saline. This was necessary for restoring important permissive functions of corticosterone which are lost following ADX (18-20). Corticosterone replacement normalizes (data not shown) basal levels of adrenocorticotropic hormone (ACTH), blood leukocyte numbers, and catecholamine hormones (21), all of which are abnormally high in ADX animals. Unlike constant replacement (via pellets or osmotic pumps), drinking water corticosterone also facilitates the normal termination of a stress-induced ACTH response (22, 23), and simulates the circadian corticosterone rhythm as animals drink at the beginning of the active period (16).

Acute stress was administered by placing animals (without squeezing or compression) in well-ventilated Plexiglas restrainers for two hours. This procedure approximates stress that is largely psychological in nature due to the perception of confinement on part of the animal (for review see: (24, 25)). Restraint activates the autonomic nervous system (21), and the hypothalamic-pituitary-adrenal axis (26-28), and results in the activation of adrenal steroid receptors throughout the body (26, 27).

The concentration and timing of each hormone administered is listed in the text and 3 0 figures. Vehicle or hormone was rapidly and gently administered via intraperitoneal (ip) injection. Corticosterone and dexamethasone (Sigma, St. Louis, Mo.) were dissolved in an aqueous solution of 2-Hydroxypropyl-b-cyclodextrin (30%, (HBC) Research Biochemicals International, Natick, Mass.). Epinephrine (Research Biochemicals International) was dissolved in sterile water.

DTH was induced by challenging the pinnae of previously sensitized rats with 2,4-dinitro-1-fluorobenzene (DNFB, Sigma, St. Louis, Mo.) or oxazalone (OXA, Sigma, St. Louis, Mo). On day 1 of sensitization, animals were anesthetized with the inhalant, methoxyflurane (Metofane; Pitman-Moore, Washington Crossing, N.J.). No anesthesia was used subsequently. An area of approximately 3×4 cm was shaved on the dorsum. The thickness of both pinnae was recorded using a constant-loading dial micrometer (Mitutoyo, Japan). On days 1 and 2 of sensitization, one hundred microliters of DNFB (1% (w/v) in 4:1, acetone:olive oil) or one hundred microliters of OXA (1.5% (w/v) in ethanol) were applied to the shaved dorsum. On day 5, baseline pinna thickness was measured. On day 6, after stress or hormone administration, the dorsal surface of the right pinnae of all animals was challenged with 50 μl of DNFB (0.5% in 4:1 acetone:olive oil) or OXA (0.75% in ethanol). Left pinnae were treated with vehicle. Pinna thickness was measured at the times shown. Measurements were made (on same relative region of pinna) gently and rapidly to avoid displacing or compressing edema fluid and changing pinna thickness. Vehicle treated (left) pinnae showed no significant change in thickness (data not shown).

The immune reaction induced using the above procedure, is characterized by swelling at the site of challenge, and by an infiltration of monocytes/macrophages and lymphocytes into the epidermis and dermis (29-31). A positive correlation between the intensity of the immune reaction and the increase in pinna thickness has been reported (32, 33). This model for skin DTH reactions has been widely used to monitor cell-mediated immune responses in vivo (30, 34).

Animals were rapidly sacrificed and cervical lymph nodes were dissected and placed in sterile PBS on ice (n=3 per treatment group). Each lymph node was subsequently weighed and disrupted between the frosted ends of two microscope slides. Suspensions of leukocytes were prepared in PBS and stored on ice for cell counting, immunofluorescent staining, and flow cytometry.

White blood cell counts and lymphocyte-neutrophil differentials were obtained on a hematology analyzer (F800, Sysmex, McGraw Park, Ill.). Specific leukocyte subtypes were measured by immunofluorescent antibody staining and subsequent analysis using three color flow cytometry (FACScan, Becton Dickinson, San Jose, Calif.). T cells were identified using the following monoclonal antibodies (Caltag, Burlingame, Calif.): CD3-FITC (1F4), CD4-PE (w3/25), CD8-TC (OX8). Briefly, cell suspensions were incubated with antibody for 20 min at room temperature, washed with PBS, and read on the FACScan with 3,000 to 5,000 events being acquired from each preparation. Appropriate isotype controls were used to set the negative criteria. Data were analyzed using Cell Quest software (Becton Dickinson, San Jose, Calif.).

For all experiments, repeated measures were made on each animal. Significant differences between timepoints within a specific leukocyte subpopulation were analyzed using the Student's t-test as a test for significant differences between means. Means that differed significantly are indicated by symbols that are defined in the figure legends. Data are expressed as mean ±SEM in all figures. A computer statistics package was used for statistical analyses (SYSTAT v5.2.1, Systat Inc., Evanston, Ill.).

Results:

Adrenal hormones mediate the stress-induced enhancement of skin DTH: In the tradition of classical endocrinology, it was hypothesized that if the stress-induced enhancement of skin DTH were mediated by adrenal hormones, adrenalectomy (ADX) would reduce or eliminate the immuno-enhancing effects of acute stress. The effects of stress on the DTH response of INTACT, sham operated were compared (SHAM), and ADX animals (FIG. 2). DTH responses of two sets of animals were examined within each treatment group. One group of INTACT, SHAM, and ADX animals (n=6 per group) was undisturbed before antigen administration (CONTROL). Another group of INTACT, SHAM, and ADX animals was restrained for 2 h immediately before antigen administration (STRESS). FIG. 1 shows that INTACT (overall significant main effect of treatment (stress), F (1, 10)=11.0, p<0.005, and for the repeated measures factor, day, F (11, 110)=29.2, p<0.001) and SHAM (overall main effect of treatment (stress), F (1, 8)=12.6, p<0.06, and for the repeated measures factor, day, F (11, 88)=11.0, p<0.001) animals showed a significant stress-induced enhancement of skin DTH, while ADX animals did not.

Immunoenhancing effects of low doses of corticosterone on skin DTH: The experiments described above showed that adrenal hormones released during stress were the major mediators of the stress-induced enhancement of skin DTH. However, the adrenal gland is the source of two principal stress hormones, the glucocorticoid, corticosterone, and the catecholamine, epinephrine. It was important to elucidate the role of each of these hormones in mediating the immuno-enhancing effects of stress. FIG. 3A shows the DTH response of ADX animals challenged with DNFB two hours after the administration of saline (control, n=5) or of a low dose of corticosterone (5 mg/kg, n=5). Having been ADX, these animals were incapable of mounting a corticosterone stress response. FIG. 3A shows that acute administration of a low dose of corticosterone to ADX animals, which mimicked the corticosterone response of adrenal intact animals, induced a significant enhancement of skin DTH.

In order to validate the finding that corticosterone, which is generally regarded as an immuno-suppressive hormone, can enhance cell-mediated immunity in vivo,the effects of administering the hormone acutely was tested, but in a non-invasive manner, to adrenal-intact animals (FIG. 3B). Vehicle (0.6% ethanol) or corticosterone (100 μg/ml or 400 μg/ml) were administered to different groups (n=5) of animals in drinking water for a period of four hours, starting two hours before, and ending two hours after the beginning of the active period of the diurnal cycle when all animals were challenged with DNFB (n=5). Since animals typically start drinking at the beginning of their active period, the animals self-administered corticosterone after which they were challenged with antigen. Plasma levels of corticosterone attained were similar to those observed during stress. FIG. 3B shows that such non-invasive corticosterone administration also resulted in a significant enhancement of skin DTH.

Immunosuppressive effects of glucocorticoid hormones on skin DTH:. While the studies described in FIG. 3 examine the effects of physiologic levels of corticosterone, the studies described in FIG. 4 examine the effects of pharmacologic treatments with glucocorticoid hormones on skin DTH. Acute administration of a high dose of corticosterone (40 mg/kg), the endogenous glucocorticoid hormone, or a low dose of dexamethasone (0.1 mg/kg), a synthetic glucocorticoid, to ADX animals significantly suppressed skin DTH (FIG. 4A, n=5). Similar results were observed following corticosterone or dexamethasone administration to adrenal intact animals (data not shown). Moreover, chronic (6 days) administration of drinking water corticosterone (400 μg/ml, n=5) significantly suppressed skin DTH (FIG. 4C). This was in contrast to acute administration (4 h) of the same dose of drinking water corticosterone which enhanced the DTH response (FIG. 3B).

Immunoenhancing effects of epinephrine on skin DTH: FIG. 5 shows the DTH response of ADX animals challenged with DNFB two hours after the administration of water (vehicle) or increasing doses (0.05, 0.25, 0.5 mg/kg) of epinephrine (n=6). Compared to vehicle-treated controls, animals treated acutely with epinephrine showed a significant dose-dependent enhancement of skin DTH.

Corticosterone and epinephrine produce an additive enhancement of skin DTH: The experiments described above showed that manipulations designed to mimic acute stress-induced changes in either corticosterone or epinephrine enhanced skin DTH. However, a physiologic stress response consists of increased plasma levels both, corticosterone and epinephrine. Therefore the potential interactions between corticosterone and epinephrine with respect to skin DTH were tested. In order to test the applicability of the previous findings to an antigen which was different from DNFB, in this experiment oxazalone (OXA) was used as the antigen. ADX animals were injected with vehicle (30% HBC), epinephrine (500 μg/kg), corticosterone (5 mg/kg), or epinephrine plus corticosterone (FIG. 6, n=6). Animals were challenged with OXA 2 h after hormone administration. Epinephrine or corticosterone administration enhanced skin DTH with the immuno-enhancement induced by corticosterone being greater than that induced by epinephrine. Moreover, simultaneous administration of epinephrine and corticosterone produced an even greater enhancement of skin DTH. These results suggest that epinephrine and corticosterone may act additively to enhance skin DTH.

Stress hormones increase T cell numbers in lymph nodes draining a skin DTH response In the present series of studies the effects of different hormone treatments were examined on the cellularity of cervical lymph nodes which drain the site (ear) of the DTH response, ADX animals were injected with vehicle (30% HBC), epinephrine (500 μg/kg), corticosterone (5 mg/kg), or epinephrine in conjunction with corticosterone (n=3). All animals were challenged with OXA 2 h after hormone administration. Absolute numbers of helper T cells (Th, CD3+CD4+), and cytolytic T cells (CTL, CD3+CD8+) were measured 48 h after the antigen exposure. FIG. 7 shows that compared with vehicle treated animals, hormone-treated animals showed significantly larger numbers of lymphocytes in cervical lymph nodes

Discussion:

Stress and stress hormones have long been regarded as being immuno-suppressive (2-7). However, a suppression of immune function under all stress conditions, would be evolutionarily maladaptive. It seems paradoxical that organisms should have evolved to suppress immune function at a time when an active immune response may be critical for survival, for example, under conditions of stress when an organism may be injured or infected by the actions of the stress-inducing agent (e.g. a predator). Another paradoxical observation is that on the one hand stress is thought to suppress immunity and increase susceptibility to infections and cancer (8, 10, 35-37), while on the other, it is thought to exacerbate inflammatory diseases (38-40) like psoriasis, asthma, and arthritis (which should be ameliorated by a suppression of immune function).

Keeping these considerations in mind, and based on the initial observations on the effects of stress and of the circadian corticosterone rhythm on leukocyte redistribution in the body (12, 13), it was demonstrated that stress has bi-directional effects on immune function such that acute stress is immuno-enhancing, while chronic stress is immuno-suppressive (1, 13, 15). The studies described herein show that hormonal manipulations which mimic acute stress produce enhancing effects on skin immunity, whereas those which mimic chronic stress suppress skin immunity.

These findings may help to explain the paradoxical situations described above. For example, under natural conditions, acute stress may serve a protective role by enhancing an immune response directed towards a wound/infection. However, a stress-induced enhancement of immune function could also be detrimental if the immune response were directed against an innocuous (poison ivy, nickel in jeweler, latex, etc.) or autoimmunogenic antigen, and this could explain the well-known stress-induced exacerbations of autoimmune diseases (38-40). It is shown herein, that chronic stress (1), and hormonal conditions which mimic chronic stress as demonstrated herein, suppress immune function. This may explain stress-induced exacerbations of infections and cancer (10, 35-37), and stress-induced suppression of wound healing (9, 41). Also, as shown high dose or prolonged administration of corticosterone, or low doses of dexamethasone, which mimic clinically-used anti-inflammatory treatments, are potently immuno-suppressive.

These studies also underline the importance of distinguishing between physiologic versus pharmacologic concentration and kinetic parameters when examining the effects of stress hormones on immune function. Thus, low doses and acute administration of corticosterone and epinephrine, which mimic acute stress, produce immuno-enhancement. Increasing the concentration of corticosterone to pharmacologic levels, or increasing the duration of corticosterone exposure to mimic levels observed during chronic stress, produces immuno-suppression. Importantly, dexamethasone, a widely used synthetic analog of corticosterone, is potently immuno-suppressive. This may be because dexamethasone bypasses several physiologic buffering mechanisms which restrict corticosterone from accessing tissues in vivo: First, dexamethasone does not bind corticosteroid binding globulin, a plasma protein which binds a large proportion of circulating corticosterone (42) and hence prevents it from activating glucocorticoid receptors in certain tissues (26, 42, 43). Second, dexamethasone has a significantly longer half-life than corticosterone (44, 45). Third, dexamethasone has a higher affinity for glucocorticoid receptors (46), and is significantly more efficient than corticosterone at activating glucocorticoid receptors in vivo (43).

The data are in line with other studies showing bi-directional effects of corticosterone on T cell proliferation (47), and stress-induced enhancements in in vitro parameters such as lymphocyte proliferation (48-51), macrophage phagocytosis (52), NK activity (53, 54), and cytokine production (55, 56). Acute stress has also been shown to enhance skin DTH (57), accelerate antigen removal (58), and increase antigen-specific antibody titres in vivo (59-62). In light of the findings (1, 12-15), stress hormones, cell adhesion molecules, cytokines, and chemokines act in concert to promote an acute stress-induced enhancement of skin immunity (1, 17). According to this model, stress hormones induce an increase in the affinity/expression of adhesion molecules on leukocytes and/or endothelial cells in compartments such as the skin and lymph nodes. This results in a selective retention of leukocytes within these compartments and increases immune surveillance. If the stress signal is followed by inflammatory mediator signals (released due to wounding or infection) at the site of leukocyte margination, leukocytes transmigrate through the endothelial lining and infiltrate the site of inflammation. Thus, a stressed organism, may mount a larger immune response by virtue of having more leukocytes at a site of challenge compared to a non-stressed animal.

In this manner, stress hormones may direct the body's soldiers (leukocytes), to exit their barracks (spleen and bone marrow), travel the boulevards (blood vessels), and take position at potential battle stations (skin, lining of gastrointestinal and urinary-genital tracks, and draining lymph nodes) (1, 12-15). Moreover, in addition to sending leukocytes to potential battle stations stress hormones may also make better equip them for battle by enhancing processes like antigen presentation, phagocytosis, and antibody production (1). Thus, a hormonal alarm signal released by the brain upon detecting a stressor, may “prepare” the immune system for potential challenges (wounding or infection) which may arise due to the actions of the stress-inducing agent (e.g. a predator or attacker). In contrast, it is likely that chronic stress suppresses immune function by decreasing leukocyte redistribution (1) and inhibiting prostaglandin synthesis and leukocyte function (11, 63).

The studies described herein are important because stress is suspected to play a role in the etiology of many diseases. Moreover, glucocorticoid as well as catecholamine hormones are prescribed for numerous clinical conditions (64, 65). A determination of the physiologic mechanisms through which stress and stress hormones enhance or suppress immune responses may help the understanding and treatment of some of these diseases. Thus, future studies will aim to facilitate the development of biomedical treatments designed to harness an individuals physiology to selectively enhance (during vaccination, wounding, infections, or cancer) or suppress (during autoimmune or inflammatory disorders) the immune response depending on what would be most beneficial for the host.

References:

1. Dhabhar, F. S. & McEwen, B. S. (1997) Brain Behavior & Immunity 11, 286-306.

2. Munck, A., Guyre, P. M. & Holbrook, N. J. (1984) Endocrine Reviews 5, 25-44.

3. Borysenko, M. & Borysenko, J. (1982) Gen. Hosp. Psychiatry 4, 59-67.

4. Khansari, D. N., Murgo, A. J. & Faith, R. E. (1990) Immunol Today 11, 170-175.

5. Kort, W. J. (1994) Adv. Neuroimmunol. 4, 1-11.

6. Maier, S. F., Watkins, L. R. & Fleshner, M. (1994) American Psychologist 49, 1004-1017.

7. Herbert, T. B. & Cohen, S. (1993) Psychosom. Med. 55, 364-379.

8. Kiecolt-Glaser, J. K., Glaser, R., Gravenstein, S., Malarkey, W. B. & Sheridan, J. (1996) PNAS USA 93, 3043-3047.

9. Marucha, P. T., Kiecolt-Glaser, J. K. & Favagehi, M. (1998) Psychosomatic Medicine 60, 362-365.

10. Sheridan, J. F. (1998) Brain, Behav., Immun. 12, 1-6.

11. Schleimer, R. P., Claman, H. N. & Oronsky, A. (1989) in Anti-inflammatory steroid action (Academic Press Inc., San Diego), pp. 564.

12. Dhabhar, F. S., Miller, A. H., Stein, M., McEwen, B. S. & Spencer, R. L. (1994) Brain Behav. Immun. 8, 66-79.

13. Dhabhar, F. S., Miller, A. P., McEwen, B. S. & Spencer, R. L. (1995b) J. Immunology 154, 5511-5527.

14. Dhabhar, F. S., Miller, A. H., McEwen, B. S. & Spencer, R. L. (1996) J. Immumology 157, 1638-1644.

15. Dhabhar, F. S. & Mcewen, B. S. (1996) J. Immunology 156, 2608-2615.

16. Dhabhar, F. S. (1996) Doctoral Dissertation, Laboratory of Neuroendocrinology, The Rockefeller University, N.Y.

17. Dhabhar, F. S. (1998) in Neuroimmunomodulation: Molecular, Integrative Systems, and Clinical Advances, eds. McCann, S. M., Lipton, J. M., Sternberg, E. M., Chrousos, G. P., Gold, P. W. & Smith, C. C. (New York Acad. Sci., New York), Vol. 840, pp. 359-372.

18. Ingle, D. J. (1954) Acta Endocrinologica 17, 172-186.

19. Ambrose, C. T. (1964) J. Exp. Med. 119, 1027-1049.

20. De Kloet, E. R. (1995) Frontiers in Neuroendocrinology 16, 416-425.

21. Kvetnansky, R., Fukuhara, K., Pacak, K., Cizza, G., Goldstein, D. S. & Kopin, I. J. (1993) Endocrinology 133, 1411-1419.

22. Akana, S. F., Jacobson, L., Cascio, C. S., Shinsako, J. & Dallman, M. F. (1988) Endocrinology 122, 1337-1342.

23. Jacobson, L., Akana, S. F., Cascio, C. S., Shinsako, J. & Dallman, M. F. (1988) Endocrinology 122, 1343-1348.

24. Berkenbosch, F., Wolvers, D. A. & Derijk, R. (1991) J. Steroid Biochem. & Mol. Biol. 40, 639-647.

25. Glavin, G. B., Par□, W. P., Sandbak, T., Bakke, H. -K. & Murison, R. (1994) Neurosci. & Biobehav. Rev. 18, 223-249.

26. Dhabhar, F. S., Miller, A. H., McEwen, B. S. & Spencer, R. L. (1995a) J. Neuroimmunol. 56, 77-90.

27. Plotsky, P. M. & Meaney, M. J. (1993) Brain Res. Molec. Brain. Res. 18, 195-200.

28. Dhabhar, F. S., McEwen, B. S. & Spencer, R. L. (1993) Brain Research 616, 89-98.

29. Vadas, M. A., Miller, J. F. A. P., Gamble, J. & Whitelaw, A. (1975) Int. Archs. Allergy Appl. Immunol. 49, 670-692.

30. Turk, J. L. (1980) Book Delayed Hypersensitivity (Elsevier, Amsterdam).

31. Malorny, U., Goebeler, M., Gutwald, J., Roth, J. & Sorg, C. (1990) Internat. Arch. Allerg. Appl. Immunol 92, 356-360.

32. Phanuphak, P., Moorhead, J. W. & Claman, H. N. (1974) J. Immunol. 112, 115-123.

33. Kimber, I. & Dearman, R. (1993) J. Pharmacol. Toxicol. Methods 29, 11-16.

34. Thorne, P. S., Hawk, C., Kaliszweski, S. D. & Guiney, P. D. (1991) Fund & Appl. Toxicol. 17, 790-806.

35. Ben-Eliyahu, S., Yirmiya, R., Liebeskind, J. C., Taylor, A. N. & Gale, R. P. (1991) Brain, Behav., Immun. 5, 193-205.

36. Cohen, S., Tyrrell, D. A. J. & Sith, A. P. (1991) New Engl J. Med 325, 606-612.

37. Glaser, R., Pearl, D. K., Kiecolt-Glaser, J. K. & Malarkey, W. B. (1994) Psychoneuroendocrinol. 19, 765-772.

38. Solomon, G. F. & Moos, R. H. (1964) Arch. Gen. Psychiat. 11, 657-669.

39. Mei-Tal, V., Meyerowitz, S. & Engel, G. (1970) Psychosom. Med 32, 67-86.

40. Thomason, B. T., Brantley, P. J., Jones, G. N., Dyer, H. R. & Morris, J. L. (1992) J. Behav. Med. 15, 215-220.

41. Padgett, D. A., Marucha, P. T. & Sheridan, J. F. (1998) Brain, Behav., Immin. 12, 64-73.

42. Suiteri, P. K., Murai, J. T., Hammond, G. L., Nisker, J. A., Raymoure, W. J. & Kuhn, R. W. (1982) Rec. Prog. Hormone. Res. 38, 457-503.

43. Spencer, R. L., Young, E. A., Choo, P. H. & McEwen, B. S. (1990) Brain Research 514, 37-48.

44. Siiteri, P. K. & Simberg, N. H. (1986) Clinics Endocrinol. & Metab. 15, 247-258.

45. Monder, C., Miroff, Y., Marandici, A. & Hardy, M. P. (1994) Endocrinol. 134, 1199-1204.

46. Svec, F. (1985) J. Steroid Biochem. 23, 669-671.

47. Wiegers, J. G., Reul, J. M. H. M., Holsboer, F. & De Kloet, E. R. (1994) Endocrinology 135, 2351-2357.

48. Wood, P. G., Karol, M. H., Kusnecov, A. W. & Rabin, B. S. (1993) Brain, Behav., Immun. 7, 121-134.

49. Rinner, I., Schauenstein, K., Mangge, H., Porta, S. & Kvetnansky, R. (1992) Brain, Behav., Immun. 6, 130-140.

50. Lysle, D. T., Cunnick, J. E. & Rabin, B. S. (1990) Brain, Behav., Immun. 4, 269-277.

51. Shurin, M. R., Zhou, D., Kusnecov, A., Rassnick, S. & Rabin, B. S. (1994) Brain, Behav., Immun. 8, 57-65.

52. Lyte, M., Nelson, S. G. & Thompson, M. L. (1990) Clin. Immunol. Immunopathol. 57, 137-147.

53. Jain, S. & Stevenson, J. R. (1991) Immunol. Invest. 20, 365-376.

54. Millar, D. B., Thomas, J. R., Pacheco, N. D. & Rollwagen, F. M. (1993) Brain, Behav., Immun. 7, 144-153.

55. Mekaouche, M., Givalois, L., Barbanel, G., Siaud, P., Maurel, D., Malaval, F., Bristow, A. F., Boissin, J., Assenmacher, I. & Ixart, G. (1994) Neuroimmunomodulation 1, 292-299.

56. Petitto, J. M., Lysle, D. T., Gariepy, J. -L. & Lewis, M. H. (1994) Brain, Behavior, and Immunity 8, 111-122.

57. Blecha, F., Barry, R. A. & Kelley, K. W. (1982) Proc. Soc. Exp. Biol. Med. 169, 239-246.

58. Sabiston, B. H., Ste Rose, J. E. M. & Cinader, B. (1978) J. Immuongenetics 5, 197-212.

59. Solomon, G. F. (1969) Int. Arch. Allergy 35, 97-104.

60. Blecha, F. & Kelley, K. W. (1981) J. Anim. Sci. 53, 439-447.

61. Cocke, R., Moynihan, J. A., Cohen, N., Grota, L. J. & Ader, R. (1993) Brain, Behav., Immun. 7, 36-46.

62. Persoons, J. H. A., Berkenbosch, F., Schornagel, K., Thepen, T. & Kraal, G. (1995) J. Allergy and Clinical Immunol. 95, 765-770.

63. Cohen, J. J. (1989) in Anti-inflammatory steroid action: basic and clinical aspects, eds. Schleimer, R. P., Claman, H. N. & Oronsky, A. (Academic Press, San Diego), pp. 110-131.

64. Haynes, R. C. J. (1990) in The pharmacological basis of experimental therapeutics, eds. Gilman, A. G., Rall, T. W., Nies, A. S. & Taylor, P. (Pergamon, N.Y.), pp. 1431-1462.

65. Hoffman, B. B. & Lefkowitz, R. J. (1990) in The pharmacological basis of experimental therapeutics, eds. Gilman, A. G., Rall, T. W., Nies, A. S. & Taylor, P. (Pergamon, N.Y.), pp. 187-241.

What is claimed is:

1. A hormone adjuvant composition comprising an amount of endogenous epinephrine, endogenous glucocorticoids, analogs thereof and combinations thereof.

2. The hormone adjuvant composition of claim 1, wherein the endogenous glucocorticoid is cortisol or corticosterone.

3. A pharmaceutical composition comprising the endogenous hormone adjuvant composition of claim 1 and a suitable carrier or diluent.

4. A vaccine comprising the endogenous hormone adjuvant composition of claim 1.

5. A therapeutic composition comprising a mixture of a therapeutically effective antigen or vaccine and the hormone adjuvant composition of claim 1.

6. The therapeutic composition according to claim 5, wherein the antigen or vaccine comprises at least one antigenic agent selected from the group consisting of: (A) viruses, bacteria, mycoplasmas, fungi, and protozoa; (B) fragments, extracts, subunits, metabolites, and recombinant constructs of (A); (C) fragments, subunits, metabolites, and recombinant constructs of mammalian proteins or glycoproteins, (D) tumor-specific antigens; (E) pathogenic organisms and non-pathogenic organisms; and (F) combinations thereof.

7. The therapeutic composition according to claim 5, wherein the antigen or vaccine comprises an antigen for a disease state selected from the group consisting of:
smallpox, yellow fever, distemper, cholera, fowl pox, scarlet fever, diphtheria, tetanus, whooping cough, influenza, rabies, mumps, measles, foot and mouth disease, poliomyelitis, viral hepatitis, influenza, diphtheria, tetanus, pertussis, measles, mumps, rubella, polio, pneumococcus, herpes, respiratory syncytial virus, haemophilus influenza type b, varicella-zoster virus or rabies.

8. A method of stimulating or enhancing an antigen-specific cell-mediated immune response which comprises administering to a subject an amount of an immunomodulator as a vaccine and a low dose of the hormone adjuvant composition of claim 1.

9. The method of claim 8, wherein the hormone adjuvant composition is administered prior to vaccination.

10. The method of claim 8, wherein the hormone adjuvant composition is administered contemporaneously with vaccination.

11. The method of claim 5, wherein the hormone adjuvant composition is administered in a vaccine.

12. A method for conferring protection against an infectious agent which comprises administering to a subject an amount of an antigen or vaccine and a low dose of the hormone adjuvant composition of claim 1.

13. A method of treating a subject with an infectious agent or cancer comprising administering to a subject an amount of the hormone adjuvant composition of claim 1.
